(19) United States
(12) Patent Application Publication
(10) Pub. No.: US 2008/0286377 A1
(43) Pub. Date: Nov. 20, 2008

(54) ANTI-RESORPTIVE BONE CEMENTS AND ALLOGENEIC, AUTOGRAFIC, AND XENOGRAFIC BONE GRAFTS

(75) Inventors: John H. Healey, New York City, NY (US); Gene R. DiResta, Yonkers, NY (US)

Correspondence Address: LAW OFFICES OF ALBERT WAI-KIT CHAN, PLLC, 141-07 20TH AVENUE, SUITE 604, WORLD PLAZA, WHITESTONE, NY, 11357, US

(73) Assignee: SLOAN-KETTERING INSTITUTE FOR CANCER RESEARCH

(21) Appl. No.: 12/135,484

(22) Filed: Jun. 9, 2008

Related U.S. Application Data
(63) Continuation of application No. 09/890,116, filed on Nov. 20, 2001, now abandoned.

(51) Int. Cl. A61K 9/14; A61K 31/683

(52) U.S. Cl. 424/501; 514/108

(57) ABSTRACT

Anti-resorptive bone cements, comprising an anti-resorptive amount of one or more anti-resorptive agents, preferably the anti-resorptive agent is a bisphosphonate. The anti-resorptive bone cements are useful for reducing bone voids and bonding prosthetic devices to bone. The invention also relates to anti-resorptive allogeneic, autografic, and xenografic bone grafts, which bone grafts comprise an anti-resorptive amount of an anti-resorptive agent such as a bisphosphonate. The anti-resorptive bone grafts are useful for reconstructive bone surgery.

This application claims the benefit of U.S. Provisional application Ser. No. 60/119,260, filed Feb. 9, 1999, incorporated by reference herein in its entirety.

### 1. FIELD OF THE INVENTION

The present invention concerns an anti-resorptive bone cement. The present invention also relates to an anti-resorptive allogeneic bone graft, an anti-resorptive autografic bone graft, and an anti-resorptive xenografic bone graft. More particularly, the present invention concerns a bone cement comprising an anti-resorptive agent, an allogeneic bone graft comprising an anti-resorptive agent, an autografic bone graft comprising an anti-resorptive agent, and a xenografic bone graft comprising an anti-resorptive agent, wherein the anti-resorptive agent is selected from the group consisting of bisphosphonates and their pharmaceutically acceptable salts or esters; salts of a Group IIIA elements; cholesterol lowering agents; bisphosphonate-chemotherapeutic agent conjugates; estrogen-bisphosphonate conjugates; and proteinaceous or hormonal anti-resorptive agents, such as estrogens, prostaglandins, and cytokines.

### 2. BACKGROUND OF THE INVENTION

### Bone Loss and Orthopaedic Implants

Progressive bone loss and pathologic fracture are major sources of skeletal pain and prosthetic failure in cancer patients.

Bone cement is used to grout most orthopaedic joint replacements. The greatest problem plaguing the durability of the implant fixation is aseptic loosening. This is induced by particulate debris shed from the implant and mediated by osteoclastic bone resorbing cells.

Thus, post-surgical bone loss associated with the use of bone cement, such as acrylic bone cement, is frequently responsible for the loosening of prosthetic implants. These osteolytic processes are associated with osteoclast activity.

Cemented orthopaedic implants undergo time dependent aseptic loosening (Martell, J. M., Berdia, S., “Determination of polyethylene wear in total hip replacement with use of the digital radiographs”, J. Bone Joint Surg. Am., 79:11, 1635-1642 (1997); Madey, S. M. Callaghan, J. J., Olejniczak, L. P., Goetz, D. D., Johnston, R. C., “Charnley total hip arthroplasty with use of improved techniques of cementing. The results after a minimum of fifteen years of follow-up”, J. Bone Joint Surg. Am., 79:1, 53-64, (1997); Neumann, L., Freund, K. G., Surensen, K. H., “Total hip arthroplasty with the charnley prosthesis in patients fifty-five years old and less. Fifteen to twenty-one year results”, J. Bone Joint Surg. Am., 78:1, 73-79, (1996); Kobayashi, S., Takaoka, K., Saito, N., Hisa, K., “Factors affecting aseptic failure of fixation after primary Charnley total hip arthroplasty. Multivariate survival analysis”, J. Bone Joint Surg. Am., 79:11, 1618-1627 (1997). These failed prostheses are painful, cause patients to lose function, necessitate surgical revision in approximately 10 percent of all arthroplasties, and represent a significant health care cost (Shanbhag, A. S., Hasselman, C. T., Rubash, H. E., “Inhibition of wear debris mediated osteolysis in a canine total hip arthroplasty model”, Clin. Orthop. Rel. Res., 344, 33-43, (1997)).

### Debris-Induced Osteolysis

Prosthetic loosening is the culmination of a series of events that begin with the formation of metal, polymethyl methacrylate (“PMMA”) cement, and polyethylene wear debris. This debris, created by normal stress between the bone-cement-prosthesis boundaries, is the inevitable consequence of normal patient movements.

In a recent review, Lewis discussed the mechanisms of bone cement induced osteolysis (Lewis G. , “Properties of Acrylic Bone Cement: State of the Art Review”, J. Biomed. Mater. Res. (Appl. Biomater.), 38, 155-182, (1997)). The debris particles find their way into the minute spaces between the bone and cement mantle.

It was reported in Roberson, J. R., Spector, M., Baggett, M. A., Kita, K., “Porous-coated femoral components in a canine model for revision arthroplasty”, J. Bone and Joint Surgery, 70A:8, 1201-1208, (1988) that debris particle size and chemical identity contribute to the virulence of the response.

Ultra-high molecular polyethylene debris has been associated with 20 percent greater bone resorption than PMMA debris. Further, debris particle size less than 10 microns, regardless of the chemical make-up, evoked the loosening process. The debris induces macrophage infiltration and a granulomatous response. Macrophage activities with the particles are linked to the biochemical environment that stimulates the formation of a periprosthetic membrane that is, in turn, associated with osteoclast mediated bone resorption. Inhibiting this osteoclast activity by anti-resorption drugs is expected to block the bone resorption step that is responsible for prosthetic loosening (Horowitz, S. M., Algan, S. A., Purdon, M. A., “Pharmacologic inhibition of particulate-induced bone resorption”, J. Biomed. Mat. Res., 31:1, 91-96, (1996). Similarly, Clohisy et al. have shown that osteoclasts mediate tumor induced local bone resorption (Clohisy, D. R., Ogilvie, C. M., Carpenter, R. J., Ramnaraine, M. L., “Localized, tumor-associated osteolysis involves the recruitment and activation of osteoclasts”, J. Orth. Res., 149:1, 2-6, (1996)).

### Anti-Resorptive Agents: Bisphosphonates

Bisphosphonates are widely used FDA-approved drugs that have been used to treat conditions characterized by excessive bone resorption. Bisphosphonates are being used experimentally to prevent morbidity from bone metastases and retard bone loss around loose orthopaedic where resorption of host bone is induced by accumulated particulate debris. Bisphosphonates are also used to treat alveolar bone resorption in dentistry.

Bisphosphonates are potent inhibitors of osteoclast activity (Mallmin et al., “Short-term effects of pamidronate disodium on biochemical markers of bone metabolism in osteoporosis—a placebo-controlled dose-finding study”, Upsala Journal of Medical Sciences, 96:3, 205-12, (1991); Fitton, A., McTavish, D., “Pamidronate: A review of its pharmacological properties and therapeutic efficacy in resorptive bond disease”, Drugs, 41:2, 289-318, (1991)) and have been used clinically to treat hypercalcemia of malignancy, Paget's disease of bone, and high turnover forms of osteoporosis. Animal studies have demonstrated the ability of bisphosphonates to prevent the development of bone metastasis (Orr, F. W., Sanchez-Sweatman, O. H. et al., “Tumor-borne interactions of skeletal metastasis”, Clin. Orthop. (US), 312, 19-33, (1995)) and reduce the number of bone events in clinical series of breast cancer, multiple myeloma, and other cancer patients.

Bisphosphonates act by blocking osteoclast function, retarding osteoblastic bone formation, and interfering with bone mineralization in a dose dependent fashion. The relative significance of the actions varies with each drug in the class. Second and third generations of bisphosphonates preferentially emphasize the desirable osteoclastic inhibitory activity (100×) and minimize or eliminate the undesirable effects.

In general, anti-resorptive bisphosphonates strongly bind to the hydroxyapatite of bone and remain bound indefinitely. The inhibition mechanism involves prevention of osteoclasts and their precursors from recognizing the bisphosphonate-hydroxyapatite matrix (Papapoulos, S. E., Hoekman, K., Lowik, C. W. G. M., Vermeij, P., Bijvoet, O. L. M.,” Application of an in vitro model and a clinical protocol in the assessment of the potency of a new bisphosphonate”, J. Bone Min. Res., 4:5, 775-782, (1989)), and by other mechanisms still being elucidated.

Bisphosphonates are used systemically to halt generalized forms of bone resorption. Experimental attempts are underway to use these drugs to treat local problems such as bone pain in monostotic fibrous dysplasia and alveolar bone resorption, but these are rare indications for systemic therapy.

Systemic administration of alendronate, a bisphosphonate, reportedly inhibited osteolysis associated with wear debris in a canine un-cemented hip arthroplasty model (Shanbhag et al., supra).

Yaffe et al. (Yaffe, A., Iztkovich, M., Earon, Y., Alt, I., Lilov, R., Binderman, I., “Local Delivery of an amino bisphosphonate prevents the resorptive phase of alveolar bone following mucoperiosteal flap surgery in rats”, J. Periodontal, 68, 884-889, (1997)) using a rat mucoperiosteal flap surgery model, administered alendronate adjacent to the animal's alveolar bone using a pellet soaked with the drug. The pellet was allowed to remain against the bone for 2 hours and was subsequently removed. The drug's, impact to the area of pellet application was monitored 21 days later. This study demonstrated that local administration significantly reduced bone resorption. Yaffe et al. previously reported that a 10-second contact of alendronate soaked pellet to alveolar bone was ineffective in preventing resorption, while systemic administration was effective.

Bisphosphonates do not interfere with the underlying mechanism of debris induced osteolysis. Bisphosphonates impede the osteoclast activity.

Ceramic hydroxyapatite dental implants for releasing bisphosphonate is discussed in Denissen, H., van Beek, E., Löwik, C, Papapoulos, S., Van den Hooff, A., “Ceramic hydroxyapatite implants for the release of bisphosphonate, Bone and Material, 25, 123-134 (1997) and Denissen, H., van Beek, E., Martinetti, R., Klein, C, van den Zer, E., Ravaglioli, A., “Net-shaped hydroxyapatite implants for release of agents modulating periodontal-like tissues”, J. Periodont Res., 32, 40-46 (1997). These publications describe impregnating bisphosphonate into inorganic ceramic implants to serve as local delivery systems to prevent bone resorption. In every case, the ceramic was formed, machined, and then soaked in a bisphosphonate solution.

Bisphosphonates block the osteoclastic bone resorption that: (1) occurs in response to particulate wear debris, the major cause of aseptic loosening of joint replacements (Kobayashi et al., supra; Hicks, D. G., Judkins, A. R., Sickel, J. Z., Rosier, R. N., Puzas, J. E., O'Keefe, R. J., “Granular histiocytosis of pelvic lymph nodes following total hip arthroplasty. The presence of wear debris, cytokine production, and immunologically activated macrophases”, J. Bone Joint Surg. Am., 78:4, 482-496, (1996)); and (2) accompanies local tumor progression and accounts for the major cause of failure in pathologic fracture treatment. Clinically, anti-resorptive agents have heretofore been used systemically to treat diseases that induce osteolytic processes. The anti-resorptive agents are distributed to bone via its capillary network in proportion to its blood flow. Following arthroplasty procedures, the amount of drug that reach sites adjacent to bone cement is lower than that adjacent to normal bone. The medullary blood supply of bone is compromised by the reaming of the femoral canal in hip replacement surgery and other arthroplasty procedures. Cementation of prostheses has other deleterious effects, including bone necrosis from the exothermic PMMA polymerization, monomer release, and impairment to the bone's capillary network (Lewis, supra). The net effect of cemented prostheses is that the local bioavailability of any drug given systemically will be relatively low. In the case of anti-resorptive agents, the drug levels at the bone-cement interface may be insufficient to adequately inhibit the osteoclasts. The durability to the response to systemic therapy is unknown.

With respect to the duration of the effect of anti-resorptive agents, repetitive systemic administrations over a long time may be needed. Local administration from a depot source has the potential to (i) deliver high titratable levels of anti-resorptive agents and (ii) provide a sustainable effect without repeat dosing.

The iontopheretic administration of bisphosphonates is disclosed in U.S. Pat. Nos. 5,735,810; 5,730,715; and 5,668,120, the entire contents of all of which is hereby incorporated by reference herein.

### Drug-Loaded PMMA Cement

Polymethyl methacrylate (PMMA) cement is effective for anchoring a prosthesis to bone. However, there are major biomechanical differences between PMMA, the prosthetic and bone. These differences, coupled with the trauma of surgery and normal post-surgical physical activity, result in the production of particulate debris, whose inevitable consequence is the cascade of events that result in implant failure.

The PMMA polymerization reaction causes some degree of osteonecrosis and disrupts bone blood flow. Thus, bisphosphonates administered systemically may not reach the affected bone-cement interface in an adequate concentration to be of significant therapeutic value. A local delivery mechanism for anti-resorption drugs to the bone surrounding the cement may be a more effective means to overcome the reduced perfusion and inhibit the wear debris induced osteoclast activity in surrounding bone.

The following categories of drugs have been impregnated in PMMA cement: (a) antibiotics, (b) cytotoxic drugs, and (c) nonsteroidal anti-inflammatory drugs. Drugs used with inorganic cements include bone morphogenetic proteins, and therapeutic peptides.

PMMA has been impregnated with a variety of drugs, including antibiotics (Duncan, C. P., Masri, B.A., “The role of antibiotic-loaded cement in the treatment of an infection after a hip replacement”, Instructional Course Lectures, 44: 305-313, (1996); Wininger, D. A., Fass, R. J., “Antibiotic-impregnated cement and beads for orthopaedic infections”, Antimicrobial Agents and Chemotherapy, 40:12, 2675-2679, (1996); Elson, R. A., Jephcott, A. E., McGechie, D. B., Verettas, D. “Antibiotic-loaded Acrylic Cement”, J. Bone Joint Surg., 59-B:2, 200-205, (1977); Baker, A. S., Greenham, L. W., “Release of Gentamicin from acrylic bone cement: Elution and diffusion studies”, J. Bone Surg., 70-A:10, 1551-1557, (1988)) and chemotherapeutic agents (Wasserlauf, S. M., Warshawsky, A., Arad-Yelin, R., Mazur, Y., Salama, R., Dekel, S., “The release of cytotoxic drugs from acrylic bone cement”, Bull. Hosp. For Joint Diseases, 53:1,68-74, (1993); Wang, H. M. Galasko, C. S. B., Crank, S., Oliver, G., Ward, C. A., “Methotrexate loaded acrylic cement in the management of skeletal Metastases: Biomechanical, Biological and Systemic Effect”, Clin. Orthoped. Rel. Res. 312, 173-186, (1995); Boland, P., Sparkes, J. M. P., Healey, J. H., “An in vivo model for delivering a chemotherapeutic agent locally to bone using polymethyl methacrylate” (Meeting abstract), Fourth Combined Meeting of the American and European Muscular Skeletal Tumor Societies, Washington, D.C., May 6-10, 1998, page 58). In one or more of the above references, PMMA was used as a support grout for the prosthetic device and depot for drugs to reduce infectious complications and the recurrence of tumor, respectively, near the implant.

Antibiotics have been mixed into cement to treat bone and periprosthetic infection. Antineoplastic drugs (such as methotrexate and cis-platinum) have been used experimentally and anecdotally for clinical indications. Iontophoresis has been reported to for encouraging incorporation of antibiotics into allografts (Megson, S., Day, R., Wood, D. J., “Iontophoresis as a means of antibiotic delivery in allograft bone”, Int. Soc. of Limb Salvage, 9th Int. Symp., Sept. 10-12, 1997, page 35, Transactions, incorporated herein by reference).

### In Vitro Studies

Characterization studies (Lewis, G., Nyman, J. S., Trieu, H. H., “Effect of mixing method on selected properties of acrylic bone cement”, J. Biomed. Mater. Res. (Appl. Biomater.), 38, 221-228, (1997); Schreurs, B. W., Spierlings, P. T. J., Huiskes, R., Slooff, T. J. J. H., “Effects of preparation techniques on the porosity of acrylic cements”, Acta. Orthop. Scanc., 59:4, 403-409, (1988); Rimnac, C. M., Wright, T. M., McGill, D. L., “The effect of centrifugation on the fracture properties of acrylic bone cements”, J. Bone Joint Surg., 68-A:2, 281-287, (1986)) using drug-PMMA constructs have demonstrated that the constructs are capable of acting as slow release drug delivery systems.

Studies performed using the several commercially available PMMA cements, e.g., “SIMPLEX®” (Howmedica, Allendale, N.J.), “PALACOS®” (Smith and Nephew, Wilimington, Del.), and others, have been performed to investigate the following characteristics:

(1) The impact of drug-level incorporation within the cement on the final biomechanical properties of the polymerized matrix. For Gentamicin (Duncan et al., supra), the addition of greater than 4.5 gm/40 gm PMMA cement weakens the resulting polymerized matrix to a level that is below the minimum standards set by the American Society of Testing and Materials (ASTM).

(2) The rate of drug release from the matrix. Drug elution tests have identified a “biphasic” elution profile, e.g., an initial, high concentration, short duration elution phase followed by a low concentration, long duration elution phase. These elution tests have also determined that the vast majority of drug remains trapped in the PMMA matrix. Elution tests performed using radiolabeled antibiotic tracers showed detectable levels of drug eluted from the matrix for periods in excess of 2 years (Elson, R. A., et al., supra). The initial rate of elution was shown to be dependent on the level of drug mixed with the PMMA cement and upon the porosity of the final matrix. Matrix porosity is directly related to drug elution rate, but inversely related to material strength. The porosity is, in turn, a function of the mixing technique and the formulation of the cement. PALACOS® cement was shown to have a higher porosity and subsequently faster drug elution rate. Centrifugation mixing of the drug-PMMA mixture resulted in the lowest porosity, “strongest” matrix, but slowest elution rate.

(3) The effect of the polymerization process on drug potency. Activity assays demonstrated that polymerization process used to obtain PMMAs, did not adversely impact or alter the therapeutic potential of the gentamicin, methotrexate, cisplatin, and other drugs.

### In Vivo Studies

Drug delivery studies in animals and humans have demonstrated that local drug levels in regions surrounding drug impregnated PMMA are significantly higher than levels measured following systemic administration. Further, the elevated local levels provided significant therapeutic advantage (Duncan et al., supra, Wininger et al., supra, Elson et al., supra and Baker et al., supra). From randomized clinical trials involving hip arthroplasties using Gentamicin-loaded PMMA, the infection rates measured at two and five year follow-up periods were shown to be significantly lower in the loaded cement group than those of the control-cement group. These studies also showed a distinct advantage of the antibiotic-loaded cement over systemic antibiotic administration.

### Lack in the Art of an In Vivo Local Delivery System for Anti-Resorptive Agents

Systemic delivery of bisphosphonates has been attempted to address the problem of osteolysis. However, heretofore there has not been an in vivo local delivery system for bisphosphonates. Citation of a reference herein shall not be construed as indicating that such reference is prior art to the present invention.

### 3. SUMMARY OF THE INVENTION

It is an object of the present invention to provide an anti-resorptive bone cement. It is a further object of the present invention to provide an anti-resorptive bone cement that is capable of bonding a prosthetic implant to bone for substantially the life of a patient.

It is a still further object of the present invention to provide an anti-resorptive bone cement useful for inhibiting debris-induced osteolysis, particularly that which follows hip arthroplasty, and providing a positive impact (e.g., promotion of bone growth and prevention of resorption) on local bone formation.

Another object of the present invention is to provide a bone cement, such as a polymethyl methacrylate (PMMA) bone cement, useful as a local drug-delivery system for an anti-resorption agent (e.g., an anti-resorptive drug) to periprosthetic bone.

It is also an object of the present invention to provide an anti-resorptive allogeneic bone graft.

It is another object of the present invention to provide an anti-resorptive autografic bone graft or an anti-resorptive xenografic bone graft.

It is still another object of the present invention to retard the rate of premature resorption of transplanted bone and to retard the rate of resorption of adjacent host bone induced by a transplanted allogeneic bone graft.

In one embodiment the invention relates to a moldable composition comprising (a) a bone cement material selected from the group consisting of an organic bone-cement dough, an inorganic bone-cement dough, and a composite bone-cement dough and (b) an anti-resorptive amount of one or more anti-resorptive agents. The anti-resorptive agent is preferably selected from the group consisting of a bisphosphonate, a pharmaceutically acceptable salt or ester thereof, a salt of a Group IIIA element, a cholesterol lowering agent; and an estrogen-bisphosphonate conjugate. More preferably, the anti-resorptive agent is a bisphosphonate selected from the group consisting of pamidronate, etidronate, and alendronate or a pharmaceutically acceptable salt or ester thereof. Preferably the bone-cement dough is an acrylic bone-cement dough, more preferably polymethyl methacrylate bone-cement dough.

In another embodiment, the invention relates to a moldable composition comprising (a) a bone-cement dough selected from the group consisting of an organic bone-cement dough, an inorganic bone-cement dough, and a composite bone-cement dough and (b) an anti-resorptive amount of one or more proteinaceous or a hormonal anti-resorptive agents.

In still another embodiment, the invention relates to a moldable composition comprising (a) a bone-cement dough selected from the group consisting of an organic bone-cement dough, an inorganic bone-cement dough, and a composite bone-cement dough and (b) a pharmaceutically effective amount of a bone-formative agent.

In yet another embodiment, the invention relates to an ex-vivo bone graft impregnated with an anti-resorptive amount of an anti-resorptive agent. Preferably, the anti-resorptive agent is selected from the group consisting of a bisphosphonate, a pharmaceutically acceptable salt or ester thereof, a salt of a Group IIIA element, a cholesterol lowering agent; and an estrogen-bisphosphonate conjugate.

In still another embodiment, the invention comprises a method of making a moldable anti-resorptive bone cement, comprising contacting a bone cement material selected from the group consisting of an inorganic bone-cement dough, an organic bone-cement dough, and a composite bone-cement dough with an anti-resorptive amount of an anti-resorptive agent. Preferably, the anti resorptive agent is selected from the group consisting of a bisphosphonate, a pharmaceutically acceptable salt or ester thereof, a salt of a Group IIIA element, a cholesterol lowering agent; a chemotherapeutic agent-bisphosphonate conjugate; and an estrogen-bisphosphonate conjugate.

In another embodiment, the invention relates to a method of making a moldable anti-resorptive bone-cement dough, comprising, contacting an organic bone-cement dough, an inorganic bone-cement dough, or a composite bone-cement dough with an anti-resorptive amount of a proteinaceous or hormonal anti-resorptive agent or with a pharmaceutically effective amount of a bone-formative agent.

In a separate embodiment, the invention comprises a method of making an anti-resorptive bone graft comprising contacting a bone graft selected from the group consisting of an allogeneic bone graft, an autografic bone graft, and a xenografic bone graft, with a fluid vehicle comprising an anti-resorptive amount of one or more anti-resorptive agents. Preferably, the anti-resorptive agent is selected from the group consisting of a bisphosphonate, a pharmaceutically acceptable salt or ester thereof, a salt of a Group IIIA element, a cholesterol lowering agent; a chemotherapeutic agent-bisphosphonate conjugate; and an estrogen-bisphosphonate conjugate.

In a further embodiment, the invention relates to a moldable composition comprising (a) a bone cement material selected from the group consisting of an organic bone-cement dough, an inorganic bone-cement dough, and a composite bone-cement dough: (b) an anti-resorptive amount of one or more anti-resorptive agents; and (c) a chemotherapeutic agent. Preferably, the anti-resorptive agent is selected from the group consisting of a bisphosphonate, a pharmaceutically acceptable salt or ester thereof, a salt of a Group IIIA element, a cholesterol lowering agent; and an estrogen-bisphosphonate conjugate. More preferably the anti-resorptive agent is a bisphosphonate and the chemotherapeutic agent preferably is doxorubicin or methotrexate.

In still another embodiment, the invention relates to a method for reducing a bone void (e.g., reducing or filling cavities or defects in bone) in a patient, in need thereof, comprising adding to the void an amount of a anti-resorptive moldable bone-cement dough composition sufficient to reduce the void. Preferably, the moldable bone-cement dough composition comprises (a) a bone cement material selected from the group consisting of an organic bone-cement dough, an inorganic bone-cement dough, and a composite bone-cement dough and (b) an anti-resorptive amount of one or more anti-resorptive agents, preferably selected from the group consisting of a bisphosphonate, a pharmaceutically acceptable salt or ester thereof, a salt of a Group IIIA element, a cholesterol lowering agent; a chemotherapeutic agent-bisphosphonate conjugate; and an estrogen-bisphosphonate conjugate.

In another embodiment, the invention comprises a bone cement kit comprising a polymer component and a liquid monomer component packaged in association with instructions, the instructions comprising: preparing a bone-cement dough comprising an anti-resorptive agent. Preferably, the polymer component or the liquid monomer component comprises the anti-resorptive agent.

The present invention may be understood more fully by reference to the detailed description, Figures, and illustrative examples, which are intended to exemplify non-limiting embodiments of the invention.

### 4. BRIEF DESCRIPTION OF THE DRAWINGS

FIG. 1 is a graph depicting compression strength vs. drug level for a pamidronate disodium-loaded PMMA cement.

FIG. 2 is a graph depicting compression strength vs. drug level for an etidronate disodium-loaded PMMA cement.

FIG. 3 is an electrophorogram of an eluted sample of etidronate disodium-impregnated PMMA.

FIG. 4 is a graph depicting the elution of pamidronate disodium from PMMA.

### 5. DETAILED DESCRIPTION OF THE INVENTION

The bone cement of the invention can be used for bonding prosthetic bones, joints, or bone grafts to skeletal tissue and reducing bone voids. Preferably, the bone cement of the invention is used in surgery, more preferably, dental or orthopedic surgery. The bone grafts of the invention can be used in place of traditional bone grafts in all known surgeries involving bone grafts or in any surgery involving skeletal tissue reconstruction wherein a bone graft is called for. For example, the bone cement and the bone grafts of the invention can be used for surgeries involving reconstruction of the hip, illium, jaw, shoulder, wrist, head, neck, face, nasal cavity, oral cavity, breast, prostate, and knee. The bone cement of the invention is especially useful for anchoring prosthetic bone and bone grafts to living bone tissue in animals, particularly mammals, more particularly humans. The bone cement of the invention is suitable for use with any prosthetic device, for example, those comprising stainless steel, titanium, cobalt chrome, ceramic, rubber, plastic, or silicone.

As used herein, the term “ex-vivo” means outside of a living organism. For instance, an ex-vivo bone graft means a bone graft outside of a patient before the bone graft is implanted in the patient by grafting the bone graft to the patient's bone. For example, a bone graft may be implanted in a patient by grafting a bone graft (e.g., and allogeneic, autografic, or xenografic bone graft) to a patient's bone during reconstructive bone surgery.

### Bone Cement

There are three basic types of bone cements, namely, organic bone cement, inorganic bone cement, and composite bone cement. Organic bone cements can comprise acrylics such as polymethyl methacrylate (PMMA) formulations, for example, “SIMPLEX®” (Howmedica, Allendale, N.J.), “PALACOS®” (Smith and Nephew, Wilminton, Del.), “Zimmer®” (Zimmer Inc., Warsaw, Ind.), and “C. M. W” (Wright Medical Technology, Arlington, Tenn.). Other acrylics useful as bone cement polymers include polymers derived from C₁-C₁₂ alkyl acrylates (e.g., methyl acrylate, ethyl acrylate, propyl acrylate, iso-propyl acrylate, H-butyl acrylate, sec-butyl acrylate, iso-butyl acrylate, tert-butyl acrylate, hexyl acrylate, heptyl acrylate, 2-heptyl acrylate, 2-ethylhexyl acrylate, 2-ethylbutyl acrylate, dodecyl acrylate, hexadecyl acrylate, 2-ethoxyethyl acrylate, isobornyl acrylate, cyclohexyl acrylate); C₁C₁₂-alkyl methacrylates (e.g., methyl methacrylate, ethyl methacrylate, propyl methacrylate, iso-propyl methacrylate, n-butyl methacrylate, sec-butyl methacrylate, iso-butyl methacrylate, tert-butyl methacrylate, hexyl methacrylate, heptyl methacrylate, 2-heptyl methacrylate, 2-ethylhexyl methacrylate, 2-ethylbutyl methacrylate, dodecyl methacrylate, hexadecyl methacrylate, 2-ethoxyethyl methacrylate, isobornyl methacrylate, cyclohexyl methacrylate); multi-functional acrylates (e.g., t-butylaminoethyl methacrylate, dimethylaminoethyl methacrylate, 2-hydroxyethyl methacrylate, 2-hydroxyethyl acrylate, glycidyl methacrylate, 1,4-butylene dimethacrylate); C₁-C₁₂ alkylene acrylates (e.g., allyl acrylate and allyl methacrylate); and copolymers of methyl acrylate or methyl methacrylate with ethylenically unsaturated compounds like acrylonitrile, butadiene, styrene, vinyl chloride, vinylidene chloride, and vinyl acetate (see, e.g., U.S. Pat. Nos. 4,791,150 and 4,064,566, incorporated herein by reference).

Inorganic cements include calcium hydroxyapatite (may be prepared according to Hayek et al Inorganic Synth. 7, 63-69 (1963)), Apatite-Wollostonite glass ceramic (Nippon Electric Glass Co., see Kawanabe et al. J. Bone Joint Surg. 80-B:3, 527-530) and hydraulic calcium phosphate (prepared as described in Bohner et al. J. Pharm. Sci. 86-5, 565-572 (1997)). Composite cements are mixtures of organic or inorganic materials or salts with organic or inorganic binders. Suitable organic and inorganic binders include the organic and inorganic bone cements described above. Suitable inorganic materials suitable for use in composite bone cements include, but are not limited, to titanium fibers and glass fibers. Organic material suitable for use in composite bone cements include but are not limited to carbon fibers and graphite. Examples of composite bone cements include graphite-in-acrylic bone cement (U.S. Pat. No. 4,064,566, incorporated herein by reference) and alumina-polylactic acid-PMMA (prepared as described in Vallet-Regi et al J. Biomed. Mater. Res. 139, 423-428 (1998), incorporated by reference herein). Salts suitable for use in composite cements include both organic and inorganic salts, for example, tricalcium phosphate particles or sodium salicylate.

Composite bone cements include, for example, a poly (propylene glycolfumarate-methyl methacrylate) matrix mixed with calcium carbonate and tricalcium phosphate particulates; a polymethyl methacrylate bone cement comprising titanium fibers; a crosslinked gelatin matrix containing tricalcium phosphate particles; glass fibers suspended in a solution of bis-phenol-A-glycidyl-methacrylate and triethylene-glycol-dimethacrylate; a composite matrix made of gelatin, water and sodium salicylate in which particulate tricalcium phosphate is entrapped; a polymethyl methacrylate bone cement comprising carbon fibers; and alumina impregnated in polymethyl (methyl methacrylate) beads.

Bone cements are generally prepared by mixing bone-cement components to give a bone-cement dough, which is particularly useful for reducing a bone void in a patient. After the bone void is reduced, the bone-cement dough can harden or cure to a bone cement. As used herein, “bone-cement components” are those materials that when admixed initially form a bone-cement dough. Bone-cement components are optionally mixed in the presence of additional chemicals, solvents, ingredients or materials. A bone-cement dough is a moldable, pliable, ductile, or deformable composition that can be manually molded by the skilled artisan to a desired shape. A bone-cement dough can be of a consistency that can be pressed into a bone void to reduce and preferably fill the bone void and conform to the void's shape. For example, a bone-cement dough can be used to reduce a bone void resulting from reconstructive bone surgery. A bone-cement dough can also be of a consistency amenable to injection into bone voids via a syringe designed for injection of bone-cement dough. For example, bone cement dough can be used to bond a prosthetic device to a bone. Here, the bone can be drilled, forming a void that can be reduced with bone-cement dough. A connecting portion of the prosthetic device can be inserted in the bone-cement dough-containing bone. The bone cement dough hardens, bonding the prosthetic device to the bone.

Preferably, the bone cement comprises an organic preferably an acrylic polymeric material. Typically, an acrylic bone cement is prepared from two components: a dry polymer component, (e.g., an acrylic powder or particulate component, such as one of the polyacrylate homopolymers and co-polymers listed above) and a liquid monomer component. The components are mixed together, preferably at room temperature, to form bone-cement dough, which is then used as desired (e.g., filling a bone void during reconstructive bone surgery or filling a bone void prior to attaching a prosthetic device to a bone)and allowed to cure to a bone cement.

Examples of suitable liquid acrylate monomers include, but are not limited to, C₁-C₁₂ alkyl acrylates (e.g., methyl acrylate, ethyl acrylate, propyl acrylate, iso-propyl acrylate, n-butyl acrylate, sec-butyl acrylate, iso-butyl acrylate, tert-butyl acrylate, hexyl acrylate, heptyl acrylate, 2-heptyl acrylate, 2-ethylhexyl acrylate, 2-ethylbutyl acrylate, dodecyl acrylate, hexadecyl acrylate, 2-ethoxyethyl acrylate, isobornyl acrylate, cyclohexyl acrylate); C₁C₁₂-alkyl methacrylates (e.g., methyl methacrylate, ethyl methacrylate, propyl methacrylate, iso-propyl methacrylate, n-butyl methacrylate, sec-butyl methacrylate, iso-butyl methacrylate, tert-butyl methacrylate, hexyl methacrylate, heptyl methacrylate, 2-heptyl methacrylate, 2-ethylhexyl methacrylate, 2-ethylbutyl methacrylate, dodecyl methacrylate, hexadecyl methacrylate, 2-ethoxyethyl methacrylate, isobornyl methacrylate, cyclohexyl methacrylate); multi-functional acrylates (e.g., t-butylaminoethyl methacrylate, dimethylaminoethyl methacrylate, 2-hydroxyethyl methacrylate, 2-hydroxyethyl acrylate, glycidyl methacrylate, 1,4-butylene dimethacrylate); C₁C₁₂ alkylene acrylates (e.g., allyl acrylate and allyl methacrylate); other ethylenically unsaturated compounds (e.g., acrylonitrile, butadiene, styrene, vinyl chloride, vinylidene chloride, and vinyl acetate); or any mixture thereof. A combination of any polymer component and liquid monomer, for example, any of those listed above, is suitable for the invention. Preferably, polymethyl methacrylate (PMMA) is the polymer component and methyl methacrylate is the monomer component. When the polymer component is an acrylic, such as PMMA, it is preferably in the form of small polymer beads or amorphous particles. When the polymer component is PMMA powder, it generally has the consistency of flour. For example, in a typical PMMA bone cement, the polymer component may comprise a mixture a particle sizes where about 65 to about 70 percent polymer particles have an average diameter of about 25 microns, and about 30 to about 35 percent of the polymer beads are about 13 to about 17 microns in diameter. The desired particle sizes and distributions are readily obtained by sifting through the appropriate screen mesh (e.g., see U.S. Pat. No. 4,341,691, incorporated by reference herein).

The composition of the liquid monomer component of a typical bone cement (e.g., see U.S. Pat. No. 4,341,691) comprises: about 95 to about 98 percent (by volume) of an acrylic monomer, preferably methyl methacrylate monomer; about 2.5 to about 3 percent (by volume) of an accelerator, such as N,N-dimethyl-p-toluidine; and about 75 ppm of a stabilizer, such as Hydroquinone. The accelerator is added to promote curing when the liquid monomer component and the polymer component are mixed at room temperature. Other examples of accelerators for use with the invention, include but are not limited to, amines, such as p-toluidine, N,N-hydroxypropyl-p-toluidine, N,N-dimethyl-p-aminophenethanol, trihexylamine, and trioctylamine; polyamines, such as N,N,N′,N′-tetramethylethylenediamine; barbituric acids, such as dimethyl barbituric acid and diethyl barbituric acid; and dimethylamino-benzene-sulphonamide; or mixtures thereof. The stabilizer advantageously prevents premature polymerization, which can occur when the liquid monomer component and the polymer component are mixed in the presence of heat, light or other materials. Example of other stabilizers suitable for use with the invention include, but are not limited to, hydroquinones and alkylated hydroquinones, such as toluhydroquinone, methyl-tert-butylhydroquinone, 2,5-di-t-butylhydroquinone 2,6-di-tert-butyl-4-methoxyphenol, 2,5-di-tert-butylhydroquinone, 2,5-di-tert-amylhydroquinone. 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, and bis(3,5-di-tert-butyl-4-hydroxyphenyl)adipate; alkylated monophenols, such as 2,6-di-tert-butyl-4-methylphenol, 2-tert-butyl-4,6-dimethylphenol and 2,6-di-tert-butyl-4-ethylphenol; alkylthiomethylphenols, such as 2,4-dioctylthiomethyl-6-tert-butylphenol and 2,4-dioctylthiomethyl-6-methylphenol; hydroxylated thiodiphenyl ethers, such as 2,2′-thiobis(6-tert-butyl-4-methylphenol) and 2,2′-thiobis(4-octylphenol); alkylidenebisphenols. such as 2,2′-methylenebis(6-tert-butyl-4-methylphenol) and 2,2′-methylenebis(6-tert-butyl-4-ethylphenol); O-, N- and S-benzyl compounds, such as 3,5,3′,5′-tetra-tert-butyl-4,4′-dihydroxydibenzyl ether and octadecyl-4-hydroxy-3,5-dimethylbenzylmercaptoacetate; and triazine compounds, such as 2,4-bis(octylmercapto-6-(3,5-di-tert-butyl-4-hydroxyanilino)-1,3,5-triazine and 2-octylmercapto-4,6-bis(3,5-di-tert-butyl-4-hydroxyanilino)-1,3,5-triazine; or any mixture thereof.

The composition of the polymer component of a preferable bone cement comprises about 80 to about 100 percent (by weight) poly methyl methacrylate, preferably about 90 percent; and optionally about 9 to about 11 percent (by weight) barium sulfate, U.S.P., preferably about 10 percent. The barium sulfate, when present, provides radiopacity so that the cement appears visible in X-ray-sensitive film when developed.

In addition, the polymer component optionally comprises a polymerization initiator, such as benzoyl peroxide, in an amount of about 0.5 to about 1 percent by weight, preferably about 0.75 percent, for initiating a free-radical polymerization process upon mixing the polymer and liquid monomer components. Preferably, small particles (e.g., about the same size or smaller than the particle size of the polymer component's particles) of the polymerization initiator are mixed with the polymer component or, when the polymer component is in the form of beads, the initiator can be incorporated therein during the bead preparation process. Other initiators suitable for use with the invention, include but are not limited to, organic peroxides, such as di-tert-butyl peroxide, dicumyl peroxide, di-tert-amyl peroxide, dibenzoyl peroxide, diacetyl peroxide, dilauroyl peroxide, succinic acid peroxide, diisononanoyl peroxide, tert-butyl peroxybenzoate, tert-butyl peroxy acetate, ethyl 3,3-di-(tert-amylperoxy)-butyrate; inorganic peroxides, such as potassium peroxydisulfate; and azo-compounds, such as 2,2′azobis[4-methoxy-2,4-dimethyl]pentanenitrile and 2,2′-azobis[2,4-dimethyl]-pentanenitrile; or mixtures thereof. Further examples of useful initiators can be found in the The Encyclopedia of Chemical Technology, I4 Kirk-Othomer (4^{th} ed. at 431-482), incorporated herein by reference).

Typically, the ratio of the liquid monomer component to the polymer component is about one milliliter of the liquid monomer component to about two grams of the polymeric component. In general, when mixing the liquid monomer and the polymer components at room temperature, the liquid monomer is added to the polymer component. The resulting mixture is stirred until a bone-cement dough is formed that preferably does not adhere to rubber gloves. The bone-cement dough is then kneaded to the consistency amenable to digital application to bone or injection into a bone void formed, for example, by drilling into a bone. A connecting portion of a prosthetic device can be inserted in the bone-cement dough-containing bone. The bone-cement dough cures, bonding the prosthetic device to the bone.

When the liquid monomer component is mixed with the polymer component, initially, the liquid monomer wets the polymer component. Since the polymer component is generally at least partially soluble in the liquid monomer, the solid polymer beads partially begin to dissolve or swell in the liquid monomer. The polymerization reaction preferably starts as soon as the two components are mixed. During the next 2 to 4 minutes, the polymerization process proceeds, changing the viscosity of the initial mixture from a syrup-like consistency (relatively lower viscosity) to a dough-like consistency (relatively higher viscosity).

PMMA, for example, can serve as a matrix appropriate to both support a prosthetic implant and deliver the anti-resorptive agent to adjacent bone osteoclast activity and thus minimize the osteolytic bone resorption.

In one embodiment, the bone-cement dough can be impregnated with one or more anti-resorptive agents. When the bone-cement dough cures, a bone cement impregnated with an anti-resorptive agent results. This embodiment is preferred when the bone cement is used for attaching a prosthesis to living bone. The bone-cement dough can be impregnated with an anti-resorptive agent by mixing the anti-resorptive agent with one or more of the bone cement's components before the components are mixed. Preferably, such mixing results in a uniform mixture. The components are then mixed according to the methods well-known in the art. The anti-resorptive agent can also be mixed into freshly prepared bone-cement dough by well-known mixing techniques. PMMA bone cements can be obtained by following known methods (e.g., U.S. Pat. Nos. 4,064,566; 4,341,691; 4,554,686; 5,334,626; 5,795,922; and 4,791,150, all of which are incorporated herein by reference) or instructions or package inserts accompanying commercial PMMA bone cement kits, e.g., “SIMPLEX®”, “PALACOS®”, “Zimmer®”, or “C.M.W®”). These bone cements can be impregnated with an anti-resorptive agent mixing the anti-resorptive agent into either the polymer component or the liquid monomer component at room temperature before the two components are mixed. It is preferable that the anti-resorptive agent is mixed into the polymer component before the polymer component is mixed with the liquid monomer component. Alternatively, the anti-resorptive agent may be impregnated in the bone cement by thoroughly mixing freshly made acrylic bone-cement dough with the anti-resorptive agent. Similarly, for inorganic and composite bone cements, the anti-resorptive agent is added to one or more of the components before preparing the bone-cement dough according to the manufacturer's instructions or according to the standard procedures well-known in the art (e.g., Denissen et al. J. Periodont. Res. 32:42-46 (1997) for calcium hydroxyapatite bone cement and U.S. Pat. No. 4,064,566 for composite bone cements, both of which are incorporated herein by reference). Alternatively, the anti-resorptive agent may be impregnated in the bone cement by thoroughly mixing freshly made bone-cement dough with anti-resorptive agent.

In another embodiment, the anti-resorptive agent can be applied to the surface of a bone-cement dough (organic, inorganic, or composite cements) by contacting pre-mixed bone-cement dough with the anti-resorptive agent. This embodiment is preferred when the bone-cement dough is used for reconstructive bone surgery or for reducing a bone void. Preferably, the bone-cement dough is formed in the shape of a sphere and contacted with the anti-resorptive agent, preferably a bisphosphonate, by rolling the dough sphere in the anti-resorptive agent, preferably in particulate form, until the external surface of the sphere is covered with the anti-resorptive agent. Preferably, the sphere is covered with the anti-resorptive agent such that it is approximately evenly distributed over the surface of the sphere. Preferably, the sphere is covered to the extent that it does not pick up more anti-resorptive agent with further rolling. For example, about 60 grams (about 50 cm³) of bone-cement dough may be prepared from a standard PMMA bone cement kit according manufacture's instructions. The resulting dough can be divided into 10 spheres (about 6 g and about 5 cm³ each) and rolled in the anti-resorptive agent, preferably a bisphosphonate, until the sphere is covered with the anti-resorptive agent evenly distributed on the sphere's surface. The sphere may then be bonded to living skeletal tissue, including teeth, during reconstructive bone surgery to act as a drug delivery device.

In a preferred embodiment, when impregnating the bone-cement dough with the anti-resorptive agent or applying the anti-resorptive agent to the surface of the bone-cement dough, anti-resorptive agent's particle size is about the same or less than the size of the bone cement's particles. When obtained commercially, bisphosphonates are generally in crystal form and should be reduced to the correct particle size. The appropriate particle size of anti-resorptive agent is readily achieved by grinding and sifting through the appropriately sized mesh screens.

The anti-resorptive agent is impregnated in the bone-cement dough or applied to the surface of the bone-cement dough in an anti-resorptive amount. As used herein, an “anti-resorptive amount” means an amount of the anti-resorptive agent sufficient to prevent loosening of the bone cement from the living bone to which it is attached for an extended period of time, preferably, about 2 to about 4 years, more preferably about 5 to about 10years, most preferably, about 11 to about 50 years and, optimally, for the life of the patient. Detecting whether the bone cement loosens from the living bone can be readily accomplished by well-known methods. For example, a radiologist or other skilled artisan can detect loosening of the bone cement by performing Gruen-zone analysis of the bone cement/bone bond and then measuring the thickness of the radiolucent line between the bone cement and the bone.

The amount of the anti-resorptive agent that is impregnated in the bone cement is dependent on the type of bone cement and anti-resorptive agent. Preferably, the anti-resorptive agent is present in an amount of about 1 microgram to about 11 grams per 60 grams of bone-cement dough, preferably, about 0.1 grams to about 10 grams per 60 grams of cement dough, and is more preferably about 0.5 grams per 60 grams of bone-cement dough. Anti-resorptive agent levels higher than these may be used until the cement's chemical or mechanical properties are compromised relative to anti-resorptive agent-free cement controls, or until local elution drug levels comprise bone remodeling processes. When the bone cement is used to attach a prosthesis to living bone, the anti-resorptive agent, preferably a bisphosphonate, in the bone-cement dough can be impregnated with an anti-resorptive agent in an amount of from about 1 microgram to about 5 milligrams of the anti-resorptive agent per 60 grams of bone-cement dough, preferably about 2 microgram to about 0.3 milligrams of the anti-resorptive agent per 60 grams of bone-cement dough.

In still another embodiment, the amount of anti-resorptive agent impregnated in the bone-cement dough is that amount used for antibiotic drugs impregnated in bone cement (e.g., Duncan et al., Instructional Course Lectures, 44, 305-313, (1996); Wininger et al., Antimicrobial Agents and Chemotherapy, 40:12, 2675-2679, (1996); Elson et al., J. Bone Joint Surg., 59-B:2, 200-205, (1977); Baker et al., J. Bone Surg., 70-A:10, 1551-1557, (1988), all of which are incorporated herein by reference).

The final level of the anti-resorptive agent impregnated in the bone-cement dough will be determined by the skilled artisan and will be subject to the nature and potency of the ant-resorptive agent; the type of bone cement dough, particularly the relationship of its mechanical strength versus the amount of anti-resorptive agent; and the physical conditions required to make the bone-cement dough (e.g., time, temperature, etc.).

When the anti-resorptive agent is to be applied to the surface of bone-cement dough (organic, inorganic, or composite cements) by contacting bone-cement dough with the anti-resorptive agent, the anti-resorptive agent is preferably contacted with the bone-cement dough until the dough surface will no longer pick up any of the anti-resorptive agent.

When loading these cements with any anti-resorptive agent, the temperature stability of the anti-resorptive agent should be considered. PMMA, for example, reaches temperatures of 70° C. during its polymerization. This is high enough to inactivate many organic molecules, e.g., proteins, etc. Another consideration is the hydration state of the anti-resorptive agent and its impact on cement polymerization or setting; for example, the PMMA polymerization reaction is adversely impacted by water incorporated within anti-resorptive salt molecules. Also, anti-resorption agents can chemically interfere with or be inactivated by the reaction chemistry of the cement during its polymerization or setting. The bone cement, which contains an anti-resorptive agent according to the present invention, can be made by pre-mixing an anti-resorptive agent, such as a bisphosphonate with, for example, a methyl methacrylate powder before adding a catalyst.

The bone cement can made with the anti-resorptive agent, such as a bisphosphonate, impregnated therein, admixed with the anti-resorptive agent, or one such as a surgeon (or other skilled artisan) can prepare the bone-cement dough at the time of use, e.g., in the operating or medical procedure room. Formation of bone-cement dough according to these methods overcomes the heretofore difficult problem of reducing the longevity of joint replacements.

After, the two components are subjected to thorough mixing, the bone-cement dough can be loaded into a syringe while still quite fluid for injection into the prepared area. Alternatively, the bone-cement dough can be kneaded for about several more minutes then it is of the proper consistency to be formed into a suitable shape for placement in the attachment site.

As is well known in the art, bone-cement dough, particularly the polymethyl methacrylate bone-cement dough, cures extremely rapidly, and unless it is used quickly, it will not flow effectively into the irregularities and projecting cavities within the prepared bone tissue.

Preferably, the bone-cement dough is added to the bone void within about three or four minutes following its preparation. Even then, the resulting bone cement to bone bond is generally stronger if the cement and prosthesis are placed into the prepared site early within this time period rather than later. However, bleeding can occur until there is sufficient counterpressure to resist it, late in the stiffening of the cement. The skilled artisan may need to balance the competing concerns of maximum cement interdigitation and minimizing bleeding at the cement-bone interface. The sooner after its preparation the bone-cement dough is applied, the less viscous it is, and the more likely that it will flow into surface irregularities and projecting cavities. The prosthesis is then advantageously held in the proper position for several more minutes while the bone-cement dough continues to harden.

### Impregnation of the Anti-Resorptive Agent in Allogeneic Bone Grafts, Autografic Bone Grafts and Xenografic Bone Grafts

The types of grafts for use in the present invention include allogeneic bone grafts, autografic bone grafts and xenografic bone grafts.

The types of allogeneic bone grafts for use in the present invention include the following:

(1) an allogeneic bone graft from another living person;

(2) an allogeneic bone graft from a cadaver, which can be obtained, for example, from a bone bank; or

(3) a freeze-dried or lypholized graft.

Cadaveric allogeneic bone grafts can be preserved by freezing to decrease immunogenicity of the bone. The process may include the use of cryopreservatives, such as ethylene glycol or DMSO, to maintain chondrocyte viability.

Allogeneic bone grafts may be also treated in several of the following ways prior to implantation:

(1) freeze drying,

(2) contact with radiation, such as 2 million rads,

(3) demineralization, such as by using 6N HCl, to leave only the protein portion of the bone, or

(4) demineralized allogeneic bone grafts in combination with vehicles such as glycerine or formulated into temperature sensitive putty to best treat surfaces or cavities requiring bone reducers.

The freeze-drying method of preserving bone grafts reduces the immunogenicity of graft material most effectively and allows grafts to be stored conveniently at room temperature in small vacuum-sealed bottles.

Fresh grafts from other living humans or frozen grafts from cadavers may contain attached soft tissue ligaments and tendons.

It is also possible according to the present invention to impregnate an autografic bone graft or a xenografic bone graft with an anti-resorptive agent. An autografic bone graft is a bone structure taken from one portion of the skeleton of an individual to be grafted to another portion of the skeleton of that individual, for example, a bone segment taken from the iliac bone of a patient to be grafted to the spine of the patient. A xenografic bone graft is a bone structure taken from one species and transplanted to a different species.

Methods that can be used to carry out the active impregnation of the anti-resorptive agent in allogeneic bone grafts, autografic bone grafts or xenografic bone grafts, so as to permanently and chemically bind the anti-resorptive agent to allogeneic bone grafts, autografic bone grafts or xenografic bone grafts include the following:

(1) Iontophoresis of bone sections.

Iontophoresis is a technique useful for delivering ions into a graft by placing a the anti-resorptive agent in a fluid vehicle, preferably an aqueous vehicle in contact with or close proximity to the graft. The fluid vehicle solution is typically carried by a first electrode pouch or receptacle. A second or dispersive electrode is placed against the graft within some proximity of the first electrode. Ions are caused to migrate from the ion-carrying medium through the graft by the application of an electrical potential or voltage of the appropriate polarity to the two electrodes. A controlled current is established by providing a sufficient voltage differential between the first and second electrodes and placing a limiting resistance or other current-limiting device elsewhere in the circuit.

Iontophoresis is used in the present invention to optimize the efficiency and effectiveness of the delivery of the anti-resorptive agent. Iontophoresis current levels and duration can be increased to attempt to drive more of the anti-resorptive agent into the bone graft matrix. Iontophoresis enhances simple diffusion of the anti-resorptive agent by the use of an electric-field gradient across the bone. This provides high local concentrations of the anti-resorptive agent to prevent premature resorption of the graft before the intended healing can occur. The procedures and apparatus for carrying out iontophoresis are described in U.S. Pat. Nos. 5,668,120, 5,730,715, and 5,735,810, all three of which are incorporated herein by reference, and can be adapted for use in the present invention. The graft is then removed from the vehicle and washed with water.

(2) A high-pressure flow of a solution of anti-resorptive agent in a fluid vehicle through bone sections by a rapid convective-diffusion.

High-pressure pumping of a solution of anti-resorptive agent, such as a bisphosphonate, through a graft matrix, such as an allogeneic bone graft, is an efficacious method for delivering anti-resorptive agent, e.g., a bisphosphonate, to internal bone regions. It is an alternative that delivers drugs primarily to surface bone. High-pressure pumping involves pumping a filtered aqueous solution of the anti-resorptive agent, such as a bisphosphonate, at a pH of approximately 7.3 and at a temperature of approximately 37° C. Such solution may include polymeric substances and/or surfactants to reduce the surface tension of the solution. This technique may require using a holding mechanism that attaches to the graft and serves as the fluid delivery point to the graft. A solution of the anti-resorptive agent is pumped via a positive pressure pump, e.g., gear, piston, etc., at pressures sufficient to drive the fluid through the graft. The pump output pressure is approximately 50 psi or more. A constant flow system is preferred, where the pump provides the requisite pressure to achieve flow through the matrix. This pressure will be influenced directly by the resistance inherent to the graft matrix. Flow is preferably slow, e.g., 5-10 milliliters/min., to facilitate the binding reaction of the anti-resorptive agent, e.g., a bisphosphonate, to the graft matrix. The flow advantageously continues for approximately 1 hour or until the concentration of the anti-resorptive agent, e.g., a bisphosphonate, in the input and output fluid streams is equal, implying bone saturation. The graft, is then removed from the vehicle and washed with water.

(3) Soaking the allograft in a solution of the anti-resorptive agent. The allograft may be soaked in a solution of the anti-resorptive agent, preferably with gentle stirring. The graft is then removed from the vehicle and washed with water.

When either of the three methods described above are used to impregnate the allograft, the concentration of the anti-resorptive agent solution should be such that the bone graft is impregnated, preferably, saturated with the anti-resorptive agent within about one to about five days. The bone graft is saturated when the anti-resorptive agent's concentration in the impregnating solution remains constant as measured by techniques well known in the art (e.g., density measurements, titration of the anti-resorptive agent, etc.). Preferably, the concentration of the anti-resorptive solution is about 0.1 grams to about 10 grams of the anti-resorptive agent per liter, more preferably, about 1 gram to about 5 grams per liter of fluid vehicle. One of skill in the art will readily be able to adjust the concentration according to the anti-resorptive agent and the fluid vehicle. The vehicle can be any fluid vehicle that is soluble in water and in which the anti-resorptive agent is soluble or partially soluble. One of skill in the art will readily choose the fluid vehicle in accordance with the anti-resorptive agent and the type and dimensions of the bone graft. Preferred vehicles include water, physiological saline or buffer solutions, glycols such as ethylene and propylene glycol, aqueous solutions of glycols, solutions of dimethyl sulfoxide and water, and mixtures thereof. The most preferred vehicle is water. The surface tension and the viscosity of the fluid vehicle may be adjusted by including one or more polymeric substances, salts, or surfactants. A large range of suitable polymeric substances, salts, and surfactants are available, and one of skill in the art will readily be able select such substances depending on the anti-resorptive agent, the fluid vehicle, and the method of impregnation.

The grafts according to the present invention, which are actively impregnated with an anti-resorptive agent as discussed hereinabove, block digestion sites of osteoclast cells and thus prevent destruction of the graft.

Thus, an embodiment of the present invention involves the pretreatment of grafts in vitro before their use in vivo in a bone grafting procedure.

### Reconstructing Damaged Bone Tissue

Bone grafting is a common procedure in skeletal reconstructive and trauma surgery to reestablish the integrity of the skeleton. It provides (1) structural support to the skeleton, principally through cortical grafts and (2) bone healing assistance to the skeleton via osteoinduction, osteoconduction, and cellular mechanisms. Various techniques are used to bridge gaps between and reduce cavities in bone. Different materials are chosen to obtain the optimal clinical combination of healing potential, biocompatibility, and convenience.

As discussed hereinabove, graft choices include an autografic bone grafts, allografts, xenografic bone grafts, or other sources. Other graft choices include naturally-occurring materials such as coral, or alloplastic materials. Examples of inorganic materials include, but are not limited to, hydroxyapatite and tricalcium phosphate synthetic implants that are readily available source material. These can be mixed with protein constituents of bone such as collagen to redcue defects and heal bones. Such materials are easily sculpted to fit the defect or impacted into a cavity.

An alternative approach is to use materials that readily conform to the defect in vivo such as cements and ceramics. Hydroxyapatite cements comprise various concentrations of calcium and phosphorus that harden in an aqueous environment at body temperature. Examples include dicalcium phosphate and tetracalcium phosphate.

The most often-used technique for reconstructing damaged bone tissue involves initially preparing the bone tissue by cutting and drilling the bone tissue so that it conforms to the shape of the securement portion of a prosthesis. Then, a number of shallow holes are generally drilled or cut into the surfaces of the bone tissue adjacent to the prosthesis in order to form projecting cavities into which bone-cement dough will flow so as to form a strong mechanical interlock between the bone cement and the bone tissue.

The prepared bone surfaces are then thoroughly cleansed of all blood, fatty marrow tissue, bone fragments, and the like, so that the bone-cement dough conforms to all of the surface irregularities of the prepared bone tissue. Finally, particularly in the case of acrylic polymeric cements, the two components of the unpolymerized bone cement are mixed.

### Anti-Resorptive Agents

As used herein the term, “anti-resorptive agent” means any material, compound, or

drag, known or to be discovered, that prevents or retards bone resorption in a patient when administered systemically or locally to the patient. Preferably, the anti-resorptive agent functions to block osteoclast activity when administered to the patient. Examples of classes of anti-resorptive agents include, but are not limited to, bisphosphonates and their pharmaceutically acceptable salts or esters; salts of a Group IIIA elements; cholesterol lowering agents; bisphosphonate-chemotherapeutic agent conjugates; estrogen-bisphosphonate conjugates; and proteinaceous or hormonal anti-resorptive agents, such as estrogens, prostaglandins, and cytokines.

As used herein, the term “cholesterol lowering agent” means any compound, material, or drug that either partially or completely interferes with the mevalonate metabolism pathway. Suitable cholesterol lowering agents for use with the invention include, but are not limited to, mevastatin, lovastatin, simvastatin, pravastatin, and fluvastatin.

As used herein, the term “bisphosphonate-conjugate” means any compound, complex, material, or drug that comprises an anti-resorptive bisphophonate associated with another material, compound, or drug via a covalent bond or an ionic bond. For example, a “bisphosphonate-chemotherapeutic agent conjugate” comprises an anti-resorptive bisphophonate associated with a chemotherapeutic agent via a covalent bond or an ionic bond and a “bisphosphonate-estrogen conjugate” comprises an anti-resorptive bisphophonate associated with an estrogen via a covalent bond or an ionic bond. Examples of bisphosphonate-estrogen conjugates are described in Bauss et al. Calcif. Tissue. Int. 59:3, 168-73 (1996) and Abstracts S478 and S479 of the Nineteenth Annual Meeting of the American Society for Bone and Mineral Research, Sep. 10-14, 1997, both of which are incorporated herein by reference. Suitable bisphosphonate-estrogen conjugates for use with the invention include 17beta-estradiol-bisphosphonate conjugates (E2-BPs), such as beta-estradiol conjugated with pamidronate, etidronate, or alendronate.

As used herein, the term “estrogen” means any female sex hormone, for example, estrone, estradiol, diethyletilbestrol, diethylstilbestol diphosphate, progesterone, norethynodrel, norethindrone, and ethnylestradiol. The term “estrogen” also encompasses estrogen like compounds, such as selective estrogen receptor modulators (SERMs). Examples of estrogen like compounds suitable for use with the invention include but are not limited to triphenylethylenes, such as tamoxifen and its derivatives, toremifene, droloxifene, and idoxifene; benzothiophenes, such as raloxifene and LY353381; chromans such as levormeloxifene; naphthalenes, such as CP336,156; and dihydronapthylenes, such as nafoxidine.

As used herein the term “prostaglandin” means a C₂₀-carboxylic acid that contains a 5 membered ring, and has the general formula:

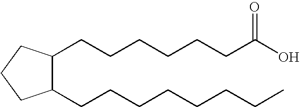

and has two or more oxygen-containing, e.g., hydroxyl, functional groups and one or more double bonds in one of the exocyclic carbon chains. Example of prostaglandins include, but are not limited, to misoprostol, prostaglandin E₂, prostaglandin F_{1α}, and prostaglandin F_{2α}.

As used herein, the term “cytokine” means a low molecular weigh hormone-like protein secreted by cells, which cells regulate the intensity and duration of the immune response. Examples of cyctokines, include but are not limited to, interleukins (e.g., I1-1 to I1-10), tumor necrosis factor-α, tuomor necrosis factor-β, and transforming growth factor-β.

It is also possible to attach drugs such as estrogen (to provide specificity of action) to a small peptide (as a vehicle to provide specificity of location) that to localize on hydroxyapatite or a matrix protein such as osteocalcin conferring specificity to bone. A protype is an (Asp)₆ conjugate with estrogen (see Abstract SA 231 of The Second Joint Meeting of The American Society for Bone and Mineral Research and The International Bone and Mineral Society, Dec. 16, 1998, incorporated herein by reference.

Any anti-resorptive agent may used in combination with one or more of any other anti-resorptive agent. For example, risedronate and prostaglandin E₂ (PGE₂), for example, see Abstract S472 “Co-treatment of prostaglandin E₂ (PGE2) and Risedronate (Ris) is equally Anabolic as PGE₂ Alone” of the Nineteenth Annual Meeting of the American Society for Bone and Mineral Research, Sep. 10-14, 1997, incorporated herein by reference.

Anti-resorptive agents for admixture with inorganic, organic, and composite bone-cement doughs in accordance with the present invention include bisphosphonates, analogs of bisphosphonates, and salts of Group IIIA elements (B, Al, Ga, In and Tl), preferably gallium salts, such as gallium nitrate, gallium chloride, gallium fluoride, gallium sulfate and gallium citrate, preferably gallium fluoride. Analogs of bisphosphonates include pharmaceutically acceptable salts and one or more phosphate esters thereof. Inorganic bone cements or composite bone cements can include an anti-resorptive amount of a proteinaceous or hormonal anti-resorptive agents, such as estrogen, prostaglandin or cytokines, and in addition thereto, or in place thereof, a pharmaceutically effective amount of a bone-formative agent can be employed such as OP-1 (BMP-7), LIM Mineralizaton Protein 1 (“LMP-1”), preferably OP-1, or a pharmaceutically effective amount of bone morphogenetic protein (“BMP”) such as BMP-2, BMP-3, BMP-4, or BMP-1, preferably BMP-2, BMP-3, or BMP-4. Any combination of proteinaceous or hormonal anti-resorptive agents and bone-formative agent may be used. As used herein, a “pharmaceutically effective amount” of a bone-formative agent or bone morphogenetic protein, means an amount that does not compromise the mechanically integrity of the bone cement, that is not toxic, and that amount that promotes bone formation.

The BMPs are novel proteins identified by Wozney J. et al. Science 242:1528-34 (1988), incorporated by reference herein, using gene cloning techniques, following earlier descriptions characterizing the biological activity in extracts of demineralized bone (Urist M. Science 150:893-99 (1965), incorporated by reference herein). Recombinant BMP-2 and BMP-4 can induce new bone formation when they are injected locally into the subcutaneous tissues of rats (Wozney J. Molec Reprod Dev. 32:160-67,(1992), incorporated by reference herein). OP-1 (also known as BP-7) can also induce new bone growth. These factors are expressed by normal osteoblasts as they differentiate, and have been shown to stimulate osteoblast differentiation and bone nodule formation in vitro as well as bone formation in vivo (Harris S. et al. J. Bone Miner. Res. 9:855-63 (1994), incorporated herein by reference). In studies of primary cultures of fetal rat calvarial osteoblasts, BMPs 1, 2, 3, 4, and 6 are expressed by cultured cells prior to the formation of mineralized bone nodules (Harris S. et al. (1994), supra). Like alkaline phosphatase, osteocalcin and osteopontin, the BMPs are expressed by cultured osteoblasts as they proliferate and differentiate. The preparation of BMPs is well-known in the art, for example, see the procedure described in U.S. Pat. No. 5,948,428 incorporated herein by reference. BMP's are available commercially from Genetics Institute, Inc (Cambridge, Mass.).

Anti-resorptive agents useful for impregnating allogeneic bone grafts, autografic bone grafts or xenografic bone grafts include one of the aforesaid bisphosphonates or analogs thereof, or said gallium salts.

Combinations of said anti-resorptive agents can be used, such as a combination of a bisphosphonate or an analog thereof or a combination of a bisphosphonate or analog thereof and a gallium salt.

Biphosphonates can be used with inorganic, composite, or organic cements. Salts of Group IIIA elements, such as gallium salts, can be used with inorganic or organic cements.

The bisphosphonate may be in its acid or salt form. Preferably the bisphosphonate is in its most clinically relevant form, for example, the commercial form marketed for and used by physicians. Non-limiting examples of bisphosphonates for use in the invention have the general structure according to formula I below:

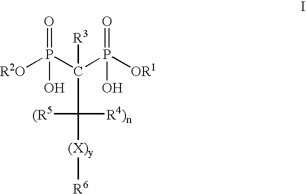

Wherein:

R¹ and R² are independently, hydrogen, an alkali metal, an alkaline earth metal, a C₁-C₄ quaternary ammonium cation, C₁C₁₀ alkyl, C₁C₁₀ unsaturated alkyl, aryl, 2-chloroethyl, 2,2,2-trichloroethyl, 2,2,2-trifluoroethyl, benzyl, or p-nitrophenyl;

R³ is hydrogen, chloro, amino, or hydroxy;

R⁴ and R⁵ are independently hydrogen, C₁-C₄ alkyl, or C₂-C₄ unsaturated alkyl;

n is an integer ranging from 1 to 7;

X is —NH—, —O—, or —S—;

y is 0 or 1; and

R⁶ is hydrogen, —NH₂, —N(R⁷)(R⁷), —N⁺(R⁷)(R⁷)(R⁷), a 5- to 7-membered aryl or cycloalkyl group, or a 5- to 7-membered heteroaryl or heterocycloalkyl group having from 1 to 3 heteroatoms one or more of which, when nitrogen, is optionally quaternary;

each R⁷ is independently hydrogen or a C₁C₄ alkyl group; and

when R⁶ is —N⁺(R⁷)(R⁷)(R⁷) or a 5- to 7-membered heteroaryl or heterocycloalkyl group having from 1 to 3 heteroatoms one or more of which is quaternary nitrogen, R⁶ is associated with a counter ion being chloride, bromide, iodide, -^{⊖}C(O)C₁-C₃ alkyl, —OH, toluenesulfonate, methylsulfonate, or trifluoromethane sulfonate.

Preferably:

R¹ and R² are independently sodium, potassium, or ammonium cation;

R³ is hydroxy;

R⁴ and R⁵ or independently hydrogen, C₁-C₄ alkyl, or C₂-C₄ unsaturated alkyl;

n is and integer ranging from 1 to3;

y is 0 or 1

R⁶ is a 5- or 6-membered heteroaryl group having 1 or 2 nitrogen atoms; and

R⁷ is a C₁-C₄ alkyl group.

More preferably:

n is 1;

the 5- or 6-membered heteroaryl group having 1 or 2 nitrogen atoms is imidazolyl or pyridyl, most preferably, 1-imidazolyl or 3-pyridyl.

According to the present invention, “substituted” means having one or more —CN, —OH, oxo, —O—C₁-C₄-alkyl, —O—C₆aryl, —CO₂H, —NH₂, —NH(C₁-C₄-alkyl), N(C₁-C₄-alkyl)₂, —NH(C₆-aryl), —N(C₆-aryl)₂, CO(C₁-C₄-alkyl), —CO₂(C₁-C₄-alkyl), —CO(C₆-aryl), or —CO₂(C₆-aryl) groups.

As used herein an “alkyl group” means a straight or branched chain monovalent radical comprised of hydrogen and carbon atoms having no unsaturation, such as methyl, ethyl, propyl, isopropyl, butyl, isobutyl, t-butyl, hexyl, heptyl, octyl, and the like which rings may be unsubstituted or substituted by one or more suitable substituents as defined above.

As used herein an “unsaturated alkyl group” means a straight or branched chain monovalent radical comprised of hydrogen and carbon atoms having one or more double bonds therein, conjugated or unconjugated, such as allyl, butenyl, pentenyl, hexenyl, heptenyl, butadienyl, pentadienyl, hexadienyl, and the like, which rings may be unsubstituted or substituted by one or more suitable substituents as defined above.

As used herein an “aryl group” means a mono- or polycyclic aromatic radical comprising carbon atoms. The aromatic ring (or rings when the aryl group is polycyclic), may be unsubstituted or substituted by one or more suitable substituents as defined above. Example of suitable aryl groups include phenyl, tolyl, indanyl, fluorenyl, indenyl, azulenyl, and naphthyl.

As used herein a “heteroaryl group” means a monocyclic aromatic ring comprising carbon atoms, preferably 3, 4, or 5 ring carbon atoms, and one or more heteroatoms selected from nitrogen, oxygen, and sulfur, which ring may be unsubstituted or substituted by one or more suitable substituents. Illustrative examples of unsubstituted heteroaryl groups include, but are not limited to furyl, pyrrolyl, imidazolyl, pyridyl, pyrazyl, pyrazolyl, pyrimidyl, thiophenyl, and phienyl.

As used herein a “cycloalkyl group” means a monocyclic radical comprising carbon atoms, preferably 5 or 6 ring carbon atoms, and having no unsaturation, which may be unsubstituted or substituted by one or more suitable substituents. Examples of cycloalkyl groups include cyclopropyl, cyclobutyl, cyclopentyl, cyclohexyl, and the like.

As used herein an “heterocycloalkyl group” means a monocyclic radical comprising carbon atoms, preferably 4 to 6 ring carbon atoms, and one or more heteroatoms selected from nitrogen, oxygen, and sulfur, and having no unsaturation, which may be unsubstituted or substituted by one or more suitable substituents. Examples of unsubstituted hetero-cycloalkyl groups include pyrrolidenyl, piperidinyl, piperazinyl, morpholinyl, and pyranyl.

Other preferred bisphosphonates useful in the present invention are represented by formula II below:

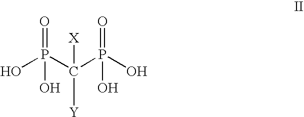

wherein X and Y independently of each other are OH, CH₃, —CH₂CH₂NH₂

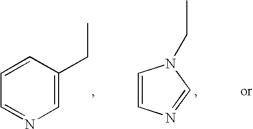

—CH₂—NH—C₁-C₆ alkyl.

Bisphosphonates for use in the present invention can be classified into two general categories: amino bisphosphonates and non-amino bisphosphonates.

In the above formula II, the OH groups may be modified to form analogs of bisphosphonates, e.g., pharmaceutically acceptable esters of bisphosphonates.

Non-limiting examples of bisphosphonates for use in the present invention include aldronic acid, etidronic acid, and pamidronic acid, which have the following formulas:

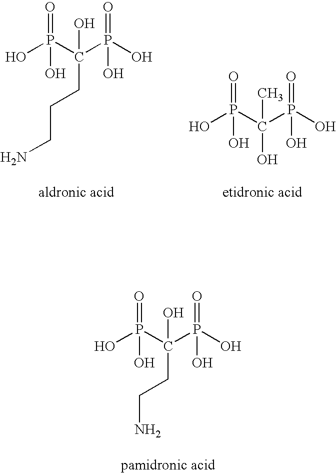

Pharmaceutically acceptable salts of aldronic, etidronic, and pamidronic acid are also useful.

Non-limiting examples of bisphosphonates salts for use in the present invention include alendronate sodium, as well as etidronate disodium, and pamidronate disodium, which have the following formulas:

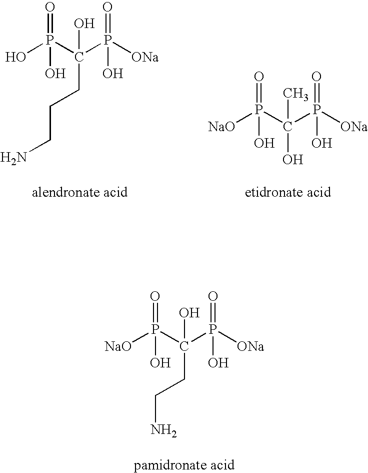

As used in herein, the term “bisphosphonate” includes both the acid form and the salt forms of the bisphosphonate.

Other bisphosphonates for use in the present invention include, but are not limited to risedronate, ibandronate, zoledronate, olpadronate, icandronate, and neridronate (6-amino-1-hydroxyexilidene-1,1-bisphosphonate);
1-hydroxyethane-1,1-bisphosphonic acid; dichloromethane bisphosphonic acid;
3-amino-1-hydroxypropane-1,1-bisphosphonic acid;
6-amino-1-hydroxyhexane-1,1-bisphosphonic acid;
4-amino-1-hydroxybutane-1,1-bisphosphonic acid;
2-(3-pyridyl)-1-hydroxyethane-1,1-bisphosphonic acid;
2-(N-imidazoyl)-1-hydroxyethane-1,1-bisphosphonic acid;
3-(N-pentyI-N-methyl amino)-1-hydroxypropane-1,1-bisphosphonic acid;
3-(N-pyrollidino)-1-hydroxypropane-1,1-bisphosphonic acid;
N-cycloheptylaminomethanebisphosphonic acid; S-(p-chlorophenyl) thiomethane-bisphosphonic acid; 4-amino-1-hydroxybutylidene-1)1-bisphosphonic acid;
(7-dihydro-1-pyrindine)methane bisphosphonic acid;
(7-dihydro-1-pyrindine)hydroxymethane bisphosphonic acid;
(6-dihydro-2-pyrindine)hydroxy-methanebisphosphonic acid;
2-(6-pyrolopyridine)-1-hydroxyethane-1,1-bisphosphonic acid; and pharmaceutically acceptable salts and esters thereof. Suitable esters include those wherein the hydrogen of one or more of the hydroxyl groups of the above bisphosphonates is replaced by C₁-C₁₀ alkyl, C₁-C₁₀ unsaturated alkyl, aryl, 2-chloroethyl, 2,2,2-trichloroethyl, 2,2,2-trifluoroethyl, benzyl, p-nitrophenyl;

Bisphosphonates for use in the present invention further include the bisphosphonates described in U.S. Pat. Nos. 5,668,120; 5,730,715; and 5,735,810, all three of which are incorporated by reference herein.

Methods useful for preparing bisphosphonates are well-known in the art and involve routing synthetic procedures. For example, other useful bisphosphonates and methods for making are described in the following patents, all incorporated by reference herein: U.S. Pat. Nos. 3,553,314; 3,683,080; 3,846,420; 3,899,496; 3,941,772; 3,957,160; 3,962,432; 3,979,385; 3,988,443; 4,113,861; 4,117,090; 4,134,969; 4,267,108; 4,304,734; 4,330,537; 4,407,761; 4,469,686; 4,578,376; 4,608,368; 4,621,077; 4,687,767; 4,687,768; 4,711,880; 4,719,203; 4,927,814; 4,990,503; and 5,019,651.

Etidronate has chemical properties that are representative of the general class of bisphosphonates. Etidronate has been used for many years to inhibit bone resorption. Its long-term use has, however, been called into question because of reports that it may impair mineralization (Mallmin et al., “Short-term effects of pamidronate on biochemical markers of bone metabolism in osteoporosis—a placebo-controlled dose-finding study”, Upsala Journal of Medical Sciences, 96:3, 205-212, (1991)).

Pamidronate has been shown to inhibit osteoclast activity and their recruitment from precursors (Mallmin et al., supra). On a molar basis, it is also one of the more potent bisphosphonates. These capabilities suggest that its action may be of longer duration than other bisphosphonates (Fitton and McTavish, supra, Mallmin et al., supra).

For impregnation into bone-cement dough, a bisphosphonate should be used without the additional buffering agents, etc., normally found in the clinical packaging.

The amount of the bisphosphonate to be utilized is an amount that will not compromise the short-term strength or the long-term durability of the bone cement or the allograft bone.

The biologic effect of the bisphosphonate should be optimized to inhibit osteoclasts enough to prevent osteolysis from particulate debris. Since the problem of particulate debris generation is time dependent, long-term delivery of bisphosphonates may be necessary to prevent the osteolytic effect. The retention of bisphosphonates in the bone matrix makes these agents excellent choices as anti-resorptive agents.

The impact of bisphosphonates on bone resorption is accessed using radiographic indicators, bone histomorphometry or density, the biochemical indicators, namely, serum alkaline phosphate (SAP) activity and level or urinary hydroxyproline (UHP) excretion. SAP activity and UHP levels are suggestive of osteoblast and osteoclast activity, respectively. An alternative indicator of osteoclast activity is the urinary level of n-telopeptide (NTX); this measure is more sensitive than UHP.

Pamidronate disodium reduces osteoclast activity, as evidenced by rapid initial fall in UHP levels. Bone formation, e.g., osteoblast activity, continues until later corrected by downward correction in osteoclast activity. This suggests that pamidronate disodium

impedes bone formation. However, its effect is secondary to control of osteoclast behavior (Fitton and McTavish, supra).

The key to the success of local delivery of bisphosphonates to the bone surrounding prosthetic implants is the duration of the remission of bone resorption following the elution of the bisphosphonates from the bone cement mantle to the adjacent bone.

The duration of the effect of pamidronate disodium can be inferred from published clinical trials using the drug for chronic bone diseases or conditions, e.g., Paget's disease, osteoporosis, or hypercalcemia of malignancy. Harnick et al. (Harnick, H. I. J., Papaoulous, S. E., Blanksma, H. J., Moolenar, A. J., Vermeij, P., Bijvoet, O. L. M., “Paget's Disease of Bone: Early and Late Responses to Three Different Modes of Treatment with Amino Hydroxy Propylidene Bisphosphonate (APD)”, British Media Journal, 295, 1301-1305, (1987)) determined in patients suffering with mild to moderate Paget's disease that administration of pamidronate for 10 days (intravenous group) to 6 months (oral group) resulted in a biochemical remission of bone resorption for a mean of 2.7 years. Further, the intravenous group responded the fastest. This study used the duration of normal SAP activity and UHP excretion as the primary indicators. Fitton and McTavish, supra, summarized the duration of the therapeutic impact of intravenous and orally administered bisphosphonate. Harnick et al. indicated that for chronic disease, single or short-term oral or intravenous administration of pamidronate disodium can reduce bone resorption for periods of several weeks to years. These observations suggest that local delivery of bisphosphonates will be effective for several years in inhibiting the debris-induced osteolysis following arthroplasty procedures.

In another embodiment of the invention, the bone cement or the bone graft of the invention may further comprise one or more chemotherapeutic agents. According to this embodiment, the term “chemotherapeutic agent” means any substance that can used to treat cancer in an animal, preferably a mammal, more preferable a human. In this embodiment, the anti-resorptive agent and the chemotherapeutic agent can be associated via a chemical bond or as a salt complex or they may be present individually in the bone cement or the bone graft. When the chemotherapeutic agent and the bisphosphonate are associated via a chemical bond or as a salt complex, the resultant compound or material is referred to herein as a “bisphosphonate-chemotherapeutic agent conjugate”. Preferably, the chemotherapeutic agent and the bisphosphonate are in the form of such a bisphosphonate-chemotherapeutic agent conjugate. The bisphosphonate may be any bisphosphonate, for example, one of those described above. Examples of suitable chemotherapeutic agents include, but are not limited to, daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, teniposide, diethylstilbestrol (DES), aldesleukin, allutamine, anastrozle, asparaginese, beg live vaccine, bicalutamide, busulfan, capecitabine, carboplain, carmustine, chlorabusil, cisplatin, cladribine, cylarabine, dscarbazine, docetaxol, doxorubicin liposmal, estramusine, etoposide, fludarabine, fluorouracil, gamcilabine, gosereine, hydroxyurea, idarubicin, itosfamide, interferon alfa, irinotecan, lauprolide actetate, levamisole, lomusline, mechlorethamine, magestrol acetate, melphalan, mesna, mitolanc, mitoxanrone, paciliaxel, pegaspergase, pentoslatin, picamycin, procarbazine, riuxlmab, straplozocin, tamoxifen, thio-tepa, thioguanine, topotecan, trastuzumab, tretinoin, vinblastine, vincristine, vinorelbine, and pharmaceutically acceptable salts thereof. For other suitable chemotherapeutic agents see, generally, The Merck Manual of Diagnosis and Therapy, 15th Ed., Berkow et al., eds., 1987, Rahway, N. J., pages 1206-1228, all of which compounds are incorporated by reference herein). Preferably, the chemotherapeutic agent is doxorubicin or methotrexate and the anti-resorptive agent is pamidronate. The preferred bisphosphonate-chemotherapeutic agent conjugate s are pamidronate/doxorubicin or pamidronate/methotrexate conjugates.

When the chemotherapeutic agent has a basic moiety such as an amine, the bisphosphonate-chemotherapeutic agent conjugate may be prepared by forming a bisphophonic acid/chemotherapeutic amine-salt complex by quenching an acidic bisphosphonate with the amine. For example, the amino function of doxorubicin may be quenched with the acid function of pamidronate to give the pamidronate/doxorubicin bisphosphonate-chemotherapeutic agent conjugate as a salt complex. Likewise, when the chemotherapeutic agent has an acidic moiety, it can be condensed with a basic group on the bisphosphonate, for example, the amino group of alendronate.

When the chemotherapeutic agent and the bisphosphonate are associated by a chemical bond, the bisphosphonate may be chemically coupled to the chemotherapeutic agent via any functional group suitable for forming a chemical bond to a suitable functional group on the chemotherapeutic agent. Such functional groups and methods for their coupling are well within the purview of one skilled in the art. Preferably, when associated by a chemical bond, the bisphosphonate is chemically bonded to the chemotherapeutic agent through one or more of its hydroxyl or amino groups. For example, the bisphosphonate-chemotherapeutic agent conjugates can be prepared by well-known chemical coupling of either an alcohol, a carboxylic acid, or an amine moiety present on the chemotherapeutic agent with the acid functionality of the bisphosphonate (e.g., see the procedures described in March, J. Advanced Organic Chemistry; Reactions Mechanisms, and Structure, 4th ed., 1992, p. p. 393-396; 401-402; and 419-421, incorporated herein by reference).

Some suitable bisphosphonate-chemotherapeutic agent conjugates for use with the invention are described in the abstract “Novel Bisphosphonate-Based Compounds for Circumventing Drug resistance in the Bone Targeting Human Tumor Cells” by A. A. Shtil; N. S. Padyukova; M. Ya. Karpeisky; and W. S. Dalton, H. Lee Moffitt Cancer Center and Research Insitute, Tampa, Fla., 33612 and V. A., Engelgardt Institute of Molecular Biology, Moscow, Russia 117984, incorporated herein by reference.

When present in bone cement, these chemotherapeutic agent-bisphosphonate drug combinations are especially useful when using the bone cement to reduce, preferably fill bone voids resulting from removal of a bony metathesis lesion, by functioning as a therapeutic support structure to deliver the chemotherapeutic agent to kill remaining tumor cells and the bisphosphonate to prevent bone resorption. Such bone cement is especially useful for reducing and patching bone voids in the vertebra after removal of tumors adjacent to the spine. Complete removal of metathesistic lesions from the spine is difficult because of the spinal cord's proximate location; thus, often, a significant portion of the tumor is left behind.

As discussed hereinabove, as an alternative to bisphosphonates or salts of Group IIIA elements, such as gallium salts, it is possible to attach drugs such as estrogen (to provide specificity of action) to a small peptide (as a vehicle to provide specificity of location) that to localize on hydroxyapatite or a matrix protein such as osteocalcin conferring specificity to bone. A protype is an (Asp)₆ conjugate with estrogen.

The function of the anti-resorptive agent with respect to grafts and bone cements is different. The function of the anti-resorptive agent in grafts is to provide chemical bonding preferably permanent chemical bonding, of the anti-resorptive agent to the graft. In contrast thereto, when an anti-resorptive agent is used with bone cements, there is a physical entrapment (e.g., impregnation) of the anti-resorptive agent in the bone cement, rather than a chemical bonding. In such resultant bone cement, the anti-resorptive agent is released by a leaching process or a passive diffusion process (generically “elution”) so as to provide an in vivo local delivery of the anti-resorptive agent to the bone.

In addition to anti-resorptive agents, the bone cements and bone grafts of the invention may further comprise one or more other biologically active substances. For example, other biologically active substances are selected from the group consisting of adrenal hormones and corticosteroids such as teracosactrin, alsactide, cortisone, cortisoneacetate, hydrocortisone, hydrocortisone alcohol, hydrocortisone acetate, hydrocortisone hemisuccinate, prednisolone, prednisoloneterbutate, 9-alphafluoroprednisolone, triamcinolone acetonide, dexamethasone phosphate, flunisolide, budesonide, toxicorol pivalate, and the like; amino acids; anorectics such as benzphetamine HCl, chlorphentermine HCl, and the like; antibiotics such as tetracycline HCl, tyrothricin, cephalosporine, aminoglycosides, streptomycin, gentamycin, leucomycin, penicillin and derivatives; erythromycin; anti-allergic agents; antibodies such as monoclonal or polyclonal antibodies against infectious diseases; anti-cholinergic agents such as atropine base; anti-emetics such as metopimazin and metochlopramide, antihistamines such as thienylperazin or anti-emetics having a regulatory effect on the motility of the intestine such as domperidom; anti-epileptics; anti-spasmolytics such as clonazepam, diazepam, nitrazepam, lorazepam, and the like; anti-histaminic and histaminic agents such as diphenhydramin HCl, chloropheniramine maleate, clemastine, histamine, prophenpyridamine maleate, chlorprophenpyridamine maleate, disodium cromoglycate, meclizine, and the like; anti-hypertensive agents such as clonidine HCl, and the like; anti-inflammatory agents (enzymatic) such as chymotrypsin, bromelain seratiopeptidase, and the like; anti-inflammatory agents (non-steroidal) such as acetaminophen, aspirin, aminopyrine, phenylbutazone, mefenamic acid, ibuprofen, diclofenac sodium, indomethacin, colchicine, probenocid, and the like; anti-inflammatory agents (steroidal) such as hydrocortisone, prednisone, fluticasone, predonisolone, triamcinolone, triamcinolone acetonide, dexamethasone, betamethasone, beclomethasone, beclomethasonedipropionate, and the like; anti-septics such as chlorhexidine HCl, hexylresorcinol, dequalinium cloride, ethacridine, and the like; anti-tussive expectorant such as sodiumcromoglycate, codeine phosphate, isoprotereol HCl, and the like; anti-viral agents such as phenyl-p-guanidino benzoate, enviroxime, and the like; beta-adrenergic blocking agents such as propranolol HCl, and the like; Blood factors such as factor VII, factor VIII and the like; bone metabolism controlling agents such as vitamin D₃ or other metabolites including combinations of 1(OH), 24(OH), and 25(OH) vitamin D₃, and the like; cardiovascular regulatory hormones such as bradykin antagonists, atrial natriuretic peptide and derivatives, angiotensin II antagonist, nitroglycerine, nifedipine, isosorbide dinitrate, propranolol, clofiliumtosylate, and the like; CNS-stimulants such as lidocaine, cocaine, and the like; diagnostic drugs such as phenolsulfonphthalein, dey T-1824, vital dyes, potassium ferrocyanide, secretin, pentagastrin, cerulein, and the like; dopaminergic agents such as bromocriptine mesylate, and the like; enzymes such as lysozyme chloride, dextranase, and the like; gastrointenstinal hormones and derivatives such as secretin, substance P, and the like; hypothalamus hormones and derivatives such as nafarelin, buserelin, zolidex, and the like, enkephalins such as metkephamid, leucine enkephalin, TRH (thyrotropin releasing hormone), and the like; local aesthetics such as benzocain, procaine, lidocaine, tetracaine, and the like; migraine treatment substances such as dihydroergotamine, ergometrine, ergotamine, pizotizin, and the like; narcotics, antagonists and analgesics such as buprenorphine, naloxone and the like; pancreatic hormones and derivatives such as insulin (hexameric/dimeric/monomeric forms), glucagon, and the like; parasympathomimetics such as nicotine, methacholine, and the like; parasympatholytics such as scopolamine, atropine, ipratropium, and the like; Parkinson disease substances such as apomorphin and the like; pituitary gland hormones and derivatives such as growth hormone (e.g. human), vasopressin and analogues, lypressin, oxytocin and analogues, and the like; protease inhibitors such as aprotinin citrate and the like; sedatives such as alprazolam, bromazepam, brotizolam, camazepam, chlordiazepeoxide, clobazam, chlorazepic acid, clonazepam, clotiazepam, cloxazolam, delorazepam, diazepam, estazolam, ethyl loflazepate, fludiazepam, flunitrazepam, flurazepam, flutazolam, halazepam, haloxazolam, ketazolam, lorazolam, lorazepam, lormetazepam, medazepam, midazolam, nimetazepam, nitrazepam, nordiazepam, oxazepam, oxazolam, pinazepam, prazepam, temazepam, tetrazepam, tofisopam, triazolam, and the like; sympathomimetics such as ephedrine, epinephrine, phenylephrine, xylometazoline, tramazoline, dopamine, dobutamine, and the like; thyroid gland hormones and derivatives such as calcitonins; vasoconstrictors such as phenylephrine HCl, tetrahydrozoline HCl, naphazoline nitrate, oxymetazoline HCl, tramazoline HCl, and the like; vasodilators such as nitroglycerin, papaverine HCl, substance P, VIP (vasoactive intestinal peptide) and the like.

### Additional Advantages of the Present Invention

The present invention thus involves the treatment, e.g., impregnation, of bone cement or allografts (particulate and large segments) with one or more anti-resorptive agents, such a bisphosphonate or a gallium salt prior to implantation to locally minimize the osteolytic processes that frequently occur following implantation of prosthetic devices or allografts as part of limb reconstructive surgery. An embodiment of the present invention thus serves to deliver an anti-resorptive agent, such as a bisphosphonate, in vivo during and following implantation. The anti-resorptive agent serves to block bone resorption to prevent osteolysis and prosthetic failure.

Local delivery of anti-resorptive agents in bone cement according to the present invention greatly increases the anti-resorptive agent concentrations at the interface and enhances the therapeutic index achievable. Bone cement impregnated with anti-resorptive agents can be used not only for implant fixation, but also as a local drug depot, delivering significantly levels of the anti-resorptive agent than those obtainable upon systemic administration. Anti-resorptive agents elute from the cement and bind to the adjacent bone matrix. The bound anti-resorptive agents inhibit regional osteoclast activity and minimize the local osteolytic processes responsible for prosthetic failure.

Anti-resorptive agents, such as bisphosphonates or salts of Group IIIA elements, such as gallium salts, for incorporation into bone-cement dough (such as PMMA bone-cement dough or hydroxyapatite bone-cement dough) or into grafts, such as allogeneic bone grafts, offer the following advantages:

(1) Anti-resorptive agents incorporated into organic, particularly an acrylic, bone cement or grafts, such as allogeneic bone grafts, retain satisfactory biomechanical characteristics.

(2) Anti-resorptive agents impregnated organic, particularly an acrylic, bone cement can serve as a slow-release depot for the drug (e.g., bisphosphonate) to adjacent bone regions.

(3) Anti-resorptive agent impregnated organic, particularly an acrylic, bone cement used in hip arthroplasty procedures significantly extend (a) the time when prosthetic loosening begins, and (b) the duration of normal levels of the bone biochemical markers serum alkaline phosphatase, N-telopeptidem, and calcium.

(4) As the anti-resorptive agent elutes out from the matrix, it binds to local bone and inhibits osteoclast activity. This in turn may reduce the need for revision surgery to correct problems associated with bone resorption.

The present invention provides an effective system that retards or prevents the rapid bone resorption that leads to failure of particulate allografts from lack of bone union or resorption and to fracture of healed, large segment grafts. This may also prevent the previously described resorption of the host bone that is induced by allograft transplants. Moreover, the local delivery of the anti-resorptive agent will induce greater bone formation stimulated from the host bone bed.

Potentially, each of the 200,000 grafts performed in the United States each year (each graft costs approximately $250-$5,000) could be pre-treated with an anti-resorptive agent by the merchant bone bank or the skilled artisan using the bone product.

The present invention also provides an improved orthopaedic implant by reducing resorption (preventing or reducing the gap between the allograft bone and the normal bone) and preventing the loss of mechanical properties, which often results in fractures.

The present invention also provides a relatively easy and inexpensive means to enhance the efficacy of allogeneic bone grafting procedures for various orthopaedic indications. The dosage of the anti-resorptive agent can be adjusted based on local requirements, creating flexibility for the skilled artisan, e.g., a surgeon. Local effects should be maximized and systemic toxicity of the anti-resorptive agents minimized. Since the graft is not vascularized, there is no oral or intravenous delivery of the anti-resorptive agent, such as a bisphosphonate, to the graft. In vitro adsorption of the anti-resorptive agent overcomes this obstacle.

The anti-resorptive agent, such as a bisphosphonate, may be permanently adsorbed to the adjacent bone, and its effect may therefore be exerted on an ongoing basis.

### EXAMPLES

### Example 1

### Evaluation of the Biomechanical Characteristics and Drug Delivery Potential of PMMA Impregnated With Bisphosphonates

Biomechanical testing was carried out on PMMA impregnated with each of etidronate disodium and pamidronate disodium. All of these tests were performed using Howmedica's Surgical SIMPLEX® PMMA cement.

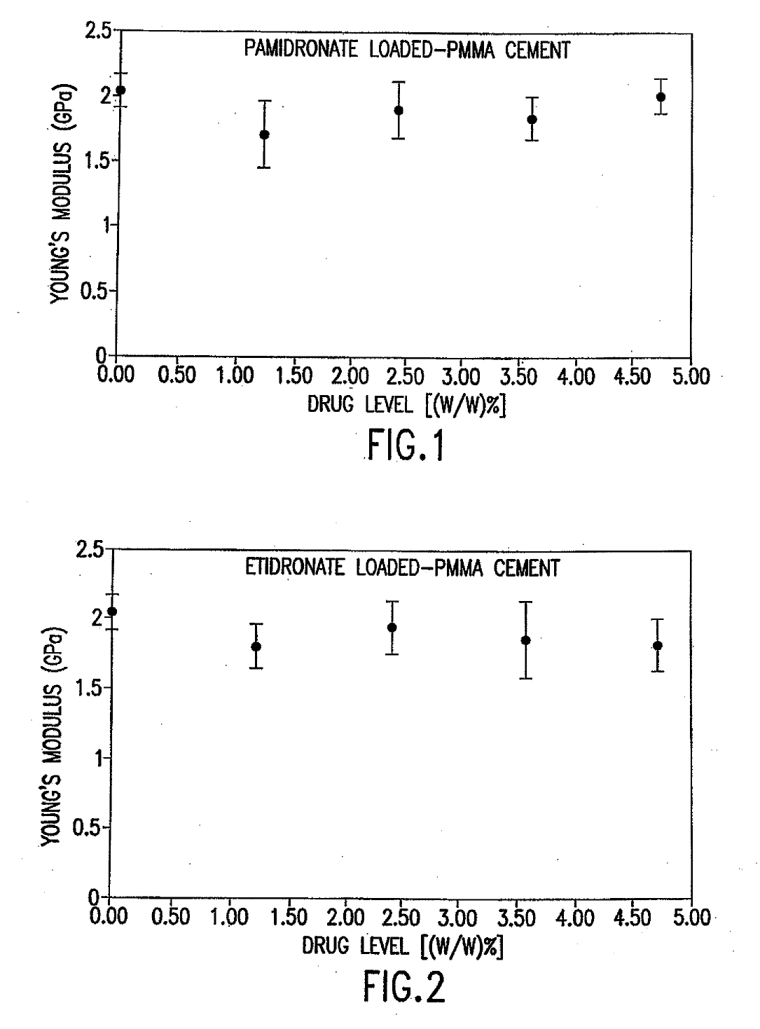

Each test was performed 10 times, as required by the United States FDA. FIGS. 1 and 2 summarize the results of compression tests on the PMMA impregnated with each of etidronate disodium and pamidronate disodium.

For all the tests, the PMMA powder (basis: 40 grams) was blended with the bisphosphonate drug using a rotary tumbler mixer for 30 minutes. Drug levels that were selected for these tests, e.g., 0.5, 1.0, 1.5, 2.0 gram/40 grams PMMA, are similar to published antibiotic drug levels (Duncan, et al., supra).

The final determination of the drug level will depend upon the elution profile of the drug from the polymerized drug-cement matrix. The blend should be such that mean elution drug levels during the initial 1 to 2 weeks of elution will approximate plasma levels following a 10-day intravenous administration of the drug. For pamidronate disodium, this level is 90 mg/70-kg patient (Fitton and McTavish, supra).

All cement-drug blends were combined with the monomer catalyst to form a dough using Howmedica's Artisan vacuum mixer. The dough was then loaded into a cement gun and injected into the various molds. The Howmedica vacuum blender produced dough with a minimum of voids. Several polymerized drug-cement matrix samples were cut on a diamond saw and revealed very few voids.

The compression and tension data shown in FIGS. 1 and 2 indicate that PMMA impregnated with etidronate disodium and pamidronate disodium do not compromise the strength of the cement. These drugs are eluted out at levels that are therapeutically effective. In addition, the local delivery of anti-resorptive agents to the bone region surrounding an implant reduces the incidence of loosening, and prolong implant longevity.

### Example 2

### Drug Level Analysis

Drug level analysis can be performed by the following techniques: capillary electrophoresis, HPLC and fluorescence spectrophotometry.

A method of analysis for pamidronate disodium and etidronate disodium samples from cement, bone and fluid samples using capillary electrophoresis (“CE”) technology is employed (Leveque, D., Gallion, C, Tarral, Monteil H., Jehl, F., “Determination of fosfomycin in biological fluids by capillary electrophoresis”, J. of Chromatography B., 655, 320-324, (1994); Olmstead, M. L., “Canine cemented total hip replacements: State of the art”, J. Small Animal Practice, 36, 395-399, (1995); Peng, S. X., Takigiku, R., Burton, D. E., Powell, L. L., “Direct Pharmaceutical Analysis of Bisphosphonates by Capillary Electrophoresis”, J. Chromatogr. B. Biomed. Sci. Appl., 709:1, 157-160, (1998)). The detection levels for these bisphosphonates are 1.0 microgram/milliliters.

Pamidronate disodium and etidronate disodium do not possess any chromaphores, and thus present a problem for routine HPLC analytical methods. Pamidronate disodium has a primary amine group that can be easily derivatized for high-pressure liquid chromatograph (“HPLC”) analysis (King, L. E., Veith, R., “Extraction and Measurement of Pamidronate disodium from Bone Samples Using Automated Pre-Column Derivatization, High Performance Liquid Chromatography and Fluorescence Detection”, Journal of Chromatography B., 678, 325-330, (1996)), however etidronate disodium does not.

The detection limit of the King and-Veith method is 0.1 microgram/milliliters. The HPLC technique is approximately ten times more sensitive than micro-electrophoresis and is used to measure pamidronate disodium levels after micro-electrophoresis detection limits are reached (this method does not work with etidronate disodium). CE bisphosphonate analysis from biological samples, such as plasma or bone, requires preparation prior to analysis; the approach used for the HPLC method for pamidronate disodium (King and Veith, supra) will be adopted.

Bisphosphonates can be highly water-soluble and can have two ionized phosphate groups. CE, a new analytical technique, performs separation by the electrical charge of the molecule.

This technique is appropriate for the analysis of such compounds and allows the indirect detection approach required for quantitating drug levels.

The separation is performed on a Hewlett Packard microelectrophoresis with sensitive flow cell using a 75 micrometers×50 centimeters bare silica capillary at −10 kv and monitored at 254 nm. The buffer consists of 1.5 mM sodium dihydrogen phosphate, 15.4 mM sodium 4-hydroxybenzoate (for indirect detection), 1.3 mM centrimide, 25 mM lithium hydroxide and 2.5 percent methanol. The retention time for etidronate disodium and pamidronate disodium are 4.7 and 5.6 minutes, respectively, and each drug can serve as the internal standard for the other. It was confirmed that there is no interference from the aqueous extracts of the bone cement or the PBS used in the elution studies. The limit of detection of the bisphosphonates using CE is approximately 1 micrograms/milliliters.

Micro-electrophoresis electrophorogram and HPLC chromatogram differences, e.g., additional peaks or retention time differences, relative to internal standards, are strongly suggestive of chemical changes.

Cement drug levels of etidronate disodium or pamidronate disodium were measured from representative samples of each drug-PMMA blend after the dough was polymerized for 24 hours. The intent of this analysis was to determine whether the drug blending technique produced a uniformly mixed powder and to determine whether the temperatures generated during the polymerization changed the chemical nature of the drug.

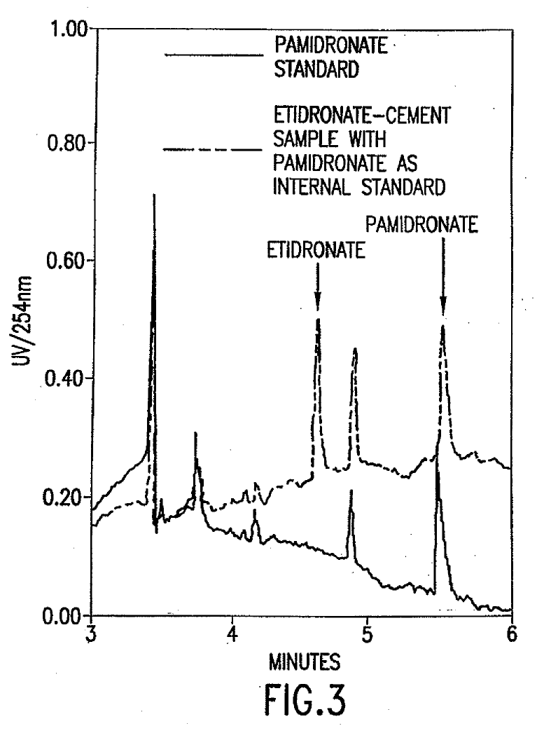

The bisphosphonate/bone containing samples were first pulverized using an SPEX Freezer Mill Model 6700. The bisphosphonates were then extracted from the powder by aqueous extraction and analyzed. The results of the analysis determined that the polymerization does not alter the structure of either bisphosphonate. The electrophorograms of the drug standard, and the drug eluted from pulverized polymerized drug-cement samples were essentially the same. Changes such as additional peaks or retention time differences, strongly suggestive of chemical changes, were not observed. Further, the analysis performed on several specimens of the drug-cement matrix demonstrated that the cement and drug were uniformly blended because the expected concentration was realized and the variance was small. FIG. 3 is the electrophorogram of an eluted sample of etidronate disodium-impregnated PMMA; pamidronate disodium serves as the internal standard for quantitation purposes.

### Example 3

### Determination of Elution Rates

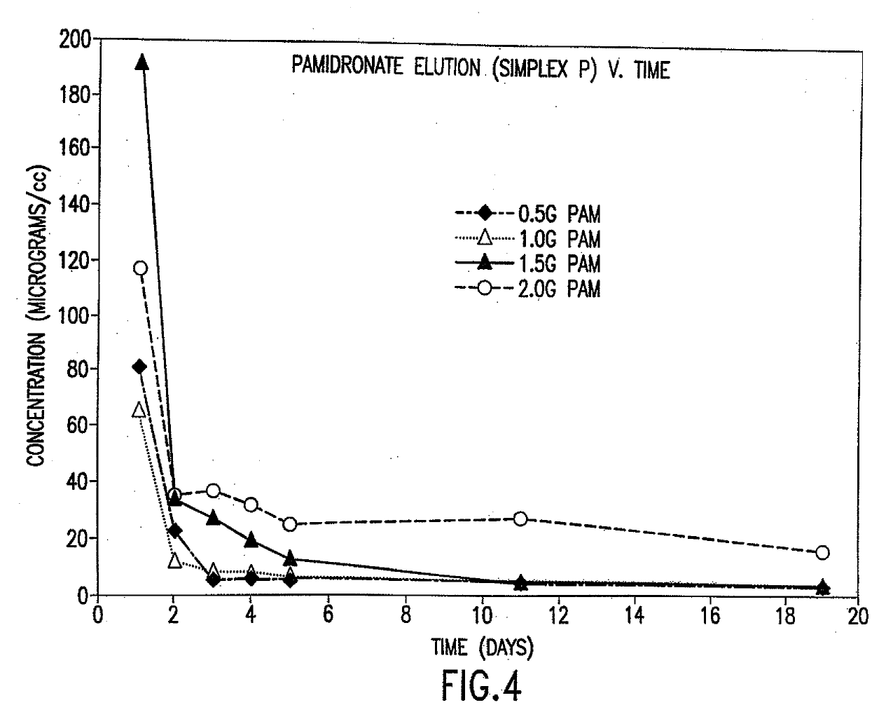

An elution study was performed using pellets of pamidronate disodium-impregnated PMMA soaked with phosphate buffered saline at 370° C. The results of this study are presented in FIG. 4. Fluid samples were withdrawn and analyzed using fluorescence spectrophotometry. This technique is a simple and rapid approach for measuring pamidronate disodium. The samples are mixed with fluoraldehyde reagent (Fischer) and read in a fluorescence spectrophotometer whose excitation wavelength is 340 nm and whose emission wavelength is 460 nm. Concentration is determined using a standard curve.

A simple mathematical model based on the “Fick” diffusion principle is used to analyze the elution data (Law, H. T., Fleming, R. H., Gilmore, M. F. X., McCarthy, I. D. and Hughes, S. P. F., “In Vitro Measurement and Computer Modeling of the Diffusion of Antibiotic in Bone Cement”, J. Biomed. Eng., 8:2, 149-155, (1986)). The model parameters are used with a subsequent mathematical model of the diffusion-adsorption process that describes the elution of drug from cement and adsorbs onto bone. These mathematical analyses, e.g., parameter estimation and simulation, are performed using the Macsyma/PDEase software package.

The present invention is not to be limited in scope by the specific embodiments disclosed in the examples which are intended as illustrations of a few aspects of the invention and any embodiments which are functionally equivalent are within the scope of this invention. Indeed, various modifications of the invention in addition to those shown and described herein will become apparent to those skilled in the art and are intended to fall within the appended claims.

1-37. (canceled)

38. A composition for local drug delivery comprising:
(a) a mixture comprising an anti-resorptive agent having a particle-size distribution which is about the same or less than that of a polymeric bone-cement component to provide for even distribution of the anti-resorptive particles throughout a polymerized bone-cement matrix after polymerization reaction; and
(b) a monomeric bone-cement component,
wherein the polymeric bone-cement component comprising the anti-resorptive agent is uniformly mixed with the monomeric bone-cement component to effect a polymerization reaction to obtain a polymerized bone-cement matrix,
wherein the anti-resorptive agent is present in an amount that does not compromise the bone cement's chemical or mechanical properties,
wherein the amount of anti-resorptive agents added to the polymeric bone-cement component does not weaken the bone-cement component or polymerized bone-cement matrix, or interfere with polymerization reaction of the bone-cement components,
wherein the polymerization of the bone cement components does not chemically interfere with or inactivate the anti-resorptive agents, and
wherein the bone-cement is polymethylmethacrylate and the anti-resorptive agent is pamidronate or etidronate or a pharmaceutically acceptable salt or ester thereof, and the bone cement comprises about 1% or more by weight of the anti-resorptive agent.

39. The composition of claim 38, wherein 65 to about 70percent of the polymeric bone-cement particles and the anti-resorptive agents have an average diameter of about 25 microns.

40. The composition of claim 38, wherein 30 to about 35percent of the polymeric bone cement particles and the anti-resorptive agents are about 13 to about 17 microns in diameter.

41. The composition of claim 38, wherein the anti-resorptive agent is present on the outer surface of the polymerized bone-cement matrix, or is uniformly distributed around the surface of the polymerized bone-cement matrix.

42. The composition of claim 38, wherein the anti-resorptive agent is impregnated throughout the polymerized bone-cement matrix after polymerization reaction.

43. The composition of claim 38 produced by the steps of: (a) mixing a polymer component with an anti-resorptive amount of an anti-resorptive agent to form a mixture; and (b) adding a liquid monomer component to the mixture.
